(19) United States
(12) Patent Application Publication
(10) Pub. No.: US 2011/0244501 A1
(43) Pub. Date: Oct. 6, 2011

(54) CANCER STEM CELLS

(75) Inventors: Petter Chu, San Diego, CA (US); Robert Peach, San Diego, CA (US)

(73) Assignee: Biogen Idec Ma Inc., Cambridge, MA (US)

(21) Appl. No.: 12/375,657

(22) PCT Filed: Aug. 2, 2007
(86) PCT No.: PCT/US07/75106

§ 371 Date: Jun. 16, 2011

Related U.S. Application Data
(60) Provisional application No. 60/821,258, filed on Aug. 2, 2006.
(60) Provisional application No. 60/895,725, filed on Mar. 19, 2007.
(60) Provisional application No. 60/950,910, filed on Jul. 20, 2007.

(51) Int. Cl. C12Q 1/02; C12N 5/095; C12N 13/00

(52) U.S. Cl. 435/29; 435/325; 435/173.9

(57) ABSTRACT

Cancer stem cell populations characterized by expression of CD44^{hi}, ABCG2, β-catenin, CD117, CD133, ALDH, VLA-2, CD166, CD201, IGFR, and/or EGF1R, and methods of isolating and using the same.

### RELATED APPLICATIONS

Priority is claimed to U.S. Provisional Application No. 60/950,910, filed Jul. 20, 2007, U.S. Provisional Application No. 60/895,725, filed Mar. 19, 2007, and U.S. Provisional No. 60/821,258, filed Aug. 2, 2006, each of which is incorporated by reference herein in its entirety.

### FIELD OF THE INVENTION

The present invention generally relates to highly tumorigenic cells, also called cancer stem cells, and methods for isolating the same. More particularly, the present invention relates to cancer stem cells expressing CD44^{hi}, ABCG2, β-catenin, CD117, CD133, ALDH, VLA-2, CD166, CD201, IGFR, and/or EGF1R. The disclosed cancer stem cell populations are useful for identification of new drugs and targets for cancer therapy, and for testing the efficacy of existing cancer drugs.

### BACKGROUND OF THE INVENTION

Colon cancer is the second leading cause of death from cancer in the Western world, where it strikes 1 out of every 20 people (Sanchez-Cespedes et al., Clin. Cancer Res., 1999, 5(9): 2450-2454). Each year colorectal cancer is responsible for over 50,000 deaths in the United States, and an estimated 500,000 deaths worldwide (Jemal et al., CA Cancer J. Clin., 2005, 55:10-30; Saunders et al., Br. J. Cancer, 2006, 95:131-138). Up to 50% of newly diagnosed patients who undergo surgical resection will develop recurrent or metastatic disease, presumably from micrometastasis to local, regional and peritoneal areas. The majority of these patients will succumb to the disease within 5 years, despite receiving standard of care adjuvant therapy such as 5-fluorouracil/leucovorin (5-FU/LV) alone or in combination with additional chemotherapeutic and/or biologic agents such as anti-VEGF (anti-vascular endothelial growth factor antibodies). Clearly, tumor cells that continue to drive the growth and spread of colon cancer, particularly after surgery and drug treatment, represent an important therapeutic target for this disease. To develop treatments that significantly increase long-term patient survival in colon cancer, cancer stem cells responsible for tumor recurrence and metastasis must be eliminated.

The normal colonic mucosa consists of a single layer of epithelial cells pock-marked with millions of mucosal invaginations or crypts. [3H]-thymidine label-retaining experiments indicate that approximately 4-6 multipotent stem cells are located at the bottom of each crypt, and which are responsible for the generation of progenitor and terminally differentiated columnar, goblet, and enteroendocrine cells lining the colon epithelium. See Potten & Loeffler, Development, 1990, 110(4): 1001-1020; Qiu et al., Epithelial Cell Biol., 1994, 3(4): 137-148. Colon stem cells are slowly dividing, relatively apoptosis-resistant cells with the capacity to undergo thousands of self-renewing asymmetric cell divisions cell divisions over their lifetime. See Potten et al., Cell Prolif., 2003, 36(3): 115-129; Cai et al., Int. J. Radiat. Biol., 1997, 71(5): 5793-5799; Potten et al., Int. J. Exp. Pathol., 1997, 78(4): 219-243; Merrit et al., J. Cell Sci. 1995, 108 (part 6):2261-2271; Lu et al., J. Pathol., 1993, 169:431-437. Each crypt is spatially organized: stem cells are located at the base of the crypt, which give rise to highly proliferative transit amplifying progenitor cells in the bottom third of the crypt. These transit amplifying cells are thought to have the ability to revert back into multipotent stem cells (Potten et al., Cell Prolif., 2003, 36(3): 115-129; Cai et al., Int. J. Radiat. Biol., 1997, 71(5): 5793-5799). The progeny of intestinal progenitors travel up the crypt, eventually losing their proliferative ability as they undergo terminal differentiation and apoptosis, and are shed into the lumen to make way for the next generation of crypt epithelial cells (Potten et al., Int. J. Exp. Pathol., 1997, 78(4): 219-243). Colon cancer originates as hyperplastic growths or aberrant crypt foci that progress into dysplastic adenomas, from which all colon cancers are thought to arise (Pinto & Clevers, Biol. Cell, 2005, 97(3): 185-196). Benign adenomas can transform into malignant tumors through a step-wise series of genetic mutations in adenomatous polyposis coli (APC) tumor suppressor, p53, k-Ras, and Smad, which is considered an adenoma-carcinoma sequence of gene expression. See Morson, Clin. Radiol., 1984, 35(6): 425-431; Fearon & Vogelstein, Cell, 1990, 61(5): 759-767. The accumulation of these mutations takes place over decades, and thus only a long-lived cell such as a colon stem cell can exist long enough to acquire the multiple mutations needed for cancer transformation (Cairns, Nature, 1975, 255(5505): 197-200).

The Wnt/β-catenin/Tcf-4 signaling pathway is essential for the maintenance of stem cells in multiple tissues (Reya, Nature, 2005, 434: 843-850). Normal intestinal epithelial stem/progenitor cells are unable to give rise to proliferative intestinal crypts in Tcf4^{-/-} mice or in the presence of a dominant negative Tcf-4 (Wielenga, Am. J. Pathol., 1999, 154: 515-523; Van de Wetering, Cell, 2002, 111: 241-250). In colon cancer, mutations in the gatekeeper gene APC lead to constitutive activation of β-catenin/Tcf-4 signaling. Colon cancer patients with wild type APC still have constitutive β-catenin activation, as a result of mutations in alternate genes, including β-catenin itself (Nathke, Ann. Rev. Cell Dev. Biol., 2004, 20:337-366). Activated nuclear β-catenin also has been shown to be important for the self-renewal of chronic myelogenous leukemia (CML) stem cells (Jamieson, N. Engl. J. Med., 2004, 351: 657-667). Collectively, these observations suggest that β-catenin could be an important link between stem/progenitor cells in normal and malignant colon tissue.

The existence of a multipotential stem cell in colon cancer is supported by experimental studies in which a subclone of the HRA-19 colon cancer cell line was expanded, injected into nude mice, and gave rise to tumors that were found to contain all colon cell lineages (Kirkland, Cancer, 1988, 61(7): 1359-1363). In addition, cells from the HT29 colon carcinoma cell line can, under appropriate conditions, differentiate in vitro into absorptive and goblet cells (Huet et al., J. Cell Biol., 1987, 105(1): 345-357).

CD34⁺ CD38⁻ cancer stem cells have been described previously in acute myelogenous leukemia (AML) (Bonnet & Dick, Nat. Med., 1997, 3(7): 730-737). CD133⁺ brain cancer cells, CD44⁺ CD24⁻ ESA⁺ breast cancer cells, and CD44⁺ prostate cancer cells have also been identified as cells with stem cell-like properties, indicating that cancer stem cells in solid tumors also exist. See Singh et al., Nature, 2004, 432(7015): 396-401; Al-Hajj et al., Proc. Natl. Acad. Sci. U.S.A., 2003, 100(7): 3983-3988; Patrawala et al., Oncogene, 2006, 25(12): 1696-1708; Kondo et al., Proc. Natl. Acad. Sci. U.S.A., 2004, 101:781-786. In all of these studies, the key criteria used to define functional cancer stem cells were high tumorigenicity, self-renewal capacity, and/or ability to recapitulate the heterogeneity of the original primary tumor.

Prospective isolation of cancer stem cells in colon cancer has not been described. The present invention provides colon cancer stem cells, and methods for identifying and isolating the same. Also provided are methods for using the disclosed cancer stem cells for developing and testing anti-cancer therapies.

### SUMMARY OF THE INVENTION

The present invention provides isolated and/or enriched cancer stem cell populations and methods of identifying the same. As described herein, the cancer stem cell populations are characterized as highly tumorigenic in vitro and in vivo, self-renewing, having an ability to differentiate, and/or apoptosis-resistance. The cancer stem cell population is alternatively described as isolated, enriched, or purified, which terms each describe a population of cells having one or more of the above-noted properties as distinguished from the properties of the source cancer cell population. Also provided are methods of prospective identification and isolation of cancer stem cells. Still further are provided methods of using the disclosed stem cell populations for testing the therapeutic efficacy of a cancer drug or candidate cancer drug.

In one aspect of the invention, an isolated stem cell may comprise at least 90% cancer stem cells, wherein the cancer stem cells (i) express CD44^{hi}, ABCG2, β-catenin, CD117, CD133, ALDH, VLA-2, CD166, CD201, IGFR, and/or EGF1R at a level that is at least 5-fold greater than differentiated cells of the same origin or non-tumorigenic cells, (ii) are tumorigenic, (iii) are capable of self-renewal, and (iv) generate tumors comprising differentiated and/or non-tumorigenic cells. A cancer stem cell population of the invention also includes an enriched cancer stem cell population derived from a tumor cell population comprising cancer stem cells and non-tumorigenic cells, wherein the cancer stem cells (i) express CD44^{hi}, ABCG2, β-catenin, CD117, CD133, ALDH, VLA-2, CD166, CD201, IGFR, and/or EGF1R at a level that is at least 5-fold greater than differentiated cells of the same origin or non-tumorigenic cells, (ii) are tumorigenic, (iii) are capable of self-renewal, (iv) generate tumors comprising non-tumorigenic cells, and (iv) are enriched at least 2-fold compared to the tumor cell population.

Cancer stem cell populations of the invention may be prepared by performing selection steps using the disclosed CD44^{hi}, ABCG2, β-catenin, CD117, CD133, ALDH, VLA-2, CD166, CD201, IGFR, and/or EGF1R markers alone, in combination, or in combination with additional positive or negative markers. For example, a method of isolating a cancer stem cell population can comprise (a) providing dissociated tumor cells, wherein a majority of the cells express CD44 at a low level, and wherein a minority of the cells express CD44 at a high level that is at least about 5-fold greater than the low level; (b) contacting the dissociated tumor cells with an agent that specifically binds to CD44; and (c) selecting cells that specifically bind to the agent of (b) to an extent that shows a high level of CD44 expression that is at least about 5-fold greater than the low level. As another example, a method of isolating cancer stem cell population can comprise (a) providing dissociated tumor cells; (b) contacting the dissociated tumor cells with an agent that specifically binds to ABCG2; and (c) selecting cells that specifically bind to the agent of (b).

The disclosed cancer stem cell populations are useful for evaluating cancer drugs and/or screening to identify new cancer drugs. As one example, the present invention provides a method of testing efficacy of a cancer drug or candidate cancer drug by (a) providing an isolated or enriched cancer stem cell population of the invention (e.g., a population expressing CD44^{hi}, ABCG2, β-catenin, CD117, CD133, ALDH, VLA-2, CD166, CD201, IGFR, and/or EGF1R as described herein); (b) contacting the cancer stem cells with a cancer drug or a candidate cancer drug; and (c) assaying a change in tumorigenic potential of the cancer stem cells in the presence of or following the contacting the cells with a cancer drug or a candidate cancer drug.

### BRIEF DESCRIPTION OF THE DRAWINGS

FIGS. 1A-1F show the expression of ABCG2 and CD44 on colon tumor cells as determined by fluorescence-activated cell sorting (FACS). ABCG2 is expressed at high levels on a small subpopulation (approximately 2%) of LS174T cells (FIG. 1A) and primary colon tumor xenograft cells (FIG. 1E). CD44 is expressed at high levels on about 17% of LS174T cells (FIG. 1B) and 24% of primary colon tumor xenograft cells (FIG. 1F). CD44 is also expressed at high levels on about 6% of SW620 cells (FIG. 1C) and 24% of HCT15 cells (FIG. 1D). Cells were sorted by expression of ABCG2 (FIGS. 1A and 1E) or CD44 (FIGS. 1B-1D and 1F) as described in Examples 1-2.

FIGS. 2A-2F show that ABCG2^{hi} and CD44^{hi} cells have significantly enriched clonogenic growth in soft agar in vitro. LS174T, HT29, and dissociated primary tumor xenograft cells were sorted by expression of ABCG2 (FIGS. 2A-2D) or CD44 (FIGS. 2E and 2F), as described in Examples 1-2.

FIGS. 2A-2B and 2D-2F show in vitro growth of colon tumor cells as assessed in soft agar assays, as described in Example 3. Data is shown as the average number of colonies per plate ±SD from at least 2 experiments.

FIG. 2C shows a representative field (40×) of soft agar plates from LS174T ABCG2^{hi} and ABCG2^{hi} sorted cells, which illustrates the increased number of large colonies derived from ABCG2^{hi} cells.

FIGS. 3A-3B are bar graphs that show CD44^{hi} cells have increased viability compared to CD44⁻ cells. CD44^{hi} and CD44⁻ cells were sorted from LS174T and SW620 cells and the primary colon tumor cell line CT1 (colon tumor 1). The isolated cells were analyzed in a cell viability assay by seeding isolated cells in 96-well plates and measuring ATP levels after 48 hours, as described in Example 3. CD44^{hi} cells were found to be more viable and to produce significantly higher levels of ATP.

FIGS. 4A-4F show a CD44^{hi} subpopulation of colon tumor cells enriched for in vitro soft agar growth and cell viability as described in Example 2. FACS analysis of CD44 expression is shown on primary colon adenocarcinoma cells from patient CT4 (FIGS. 4A-4B), passaged CT4 xenograft tumor cells (FIGS. 4C-4D), and passaged CT5 xenograft tumor (FIGS. 4E-4F). CD44 was detected with a pan-CD44 antibody that detects all CD44 isoforms. Staining with an isotype antibody was used to set the CD44⁻ cell gate. The CD44^{hi} gate was then set to capture cells with a fluorescence intensity at least ½ log higher than the CD44⁻ gate.

FIGS. 5A-5F show FACs analysis of CT5 primary colon tumor xenografts passaged through three serial transfers of 1000 CD44^{hi} cells. FIGS. 5A-5C show CD44 stained samples, and FIGS. 5D-5F show matched isotype controls for each sample used to gate the CD44⁻ population.

FIGS. 6A-6F show that CD44^{hi} and ABCG2^{hi} primary colon tumor cells are highly tumorigenic in vivo. Primary colon tumor xenograft cells from patient CT2 (FIGS. 6A-6C and 6E-6F) and patient CT3 (FIG. 6D) were depleted of dead cells (PI⁺, propidium iodide positive cells) and mouse cells (H2D^{d} and H2K^{d} positive cells), and sorted by high expression of CD44 or ABCG2, prior to subcutaneous implantation into Scid/Bg mice. Mice were monitored for tumor formation approximately once or twice per week. See Example 4.

FIG. 6A is a scatter plot of individual tumors at day 26, which show individual tumor volumes of mice from 5 groups implanted with 10,000 cells from isolated CD44^{hi}, CD44⁻, ABCG2^{hi}, ABCG2⁻, or unsorted live CT2 cells (n=9 mice/group). Unsorted cells lacked staining with propidium iodide and also lacked expression of H2Dd and H2 Kd. Horizontal bar represents the mean tumor volume for each group (n=9).

FIGS. 6B-6F are in vivo tumor growth curves measuring mean tumor volume over time. Tumor growth curves show data from mice implanted with 10,000 or 1,000 cells from CT2 or CT3 tumor xenografts, as indicated. Mean tumor volume at each time point was calculated using the equation length×width²/2. Mice were followed until they had to be euthanized due to tumor size, ulceration or visible signs of illness, or until no change in tumor size was observed for several weeks. Values shown are the mean+/−SEM (n=9 in all experiments, with the exception of ABCG2^{hi} 10,000 cell groups in FIGS. 6A and 6E, where n=8, and FIG. 6D, where n=5). Asterisk indicates that mean tumor volume is significantly different than the matched control at the same time point (p<0.05, paired student's t test).

FIGS. 7A-7B show a cell cycle analysis comparing CD44^{hi} and CD44⁻ sorted CT5 primary colon tumor xenograft cells. Similar results were seen for CT2 and CT4.

FIG. 8 shows that isolated CD44^{hi} colon tumor cells have self-renewal capacity and generate tumors having both CD44⁻ and CD44^{hi} cells. CD44^{hi} primary colon tumor xenograft tumor cells were sorted by flow cytometry (left panel shows CD44 expression in the parental primary xenograft) and 100 cells were implanted per mouse as described in Example 4. The parental primary xenograft is derived from several passages of whole tumor fragments obtained from the original patient tumor. A tumor derived from 100 CD44^{hi} cells was harvested, dissociated, and analyzed by flow cytometry for CD44 expression (first generation or 1° CD44-derived tumor). Serial transplantation of 100 CD44^{hi} or CD44⁻ cells from the 1° CD44-derived tumor was then performed. See Example 5. Mice serially transplanted with 100 CD44^{hi} cells formed tumors in all 5 mice, and these tumors showed the same CD44 expression profile (2° CD44-derived tumor). In third and fourth serial transplantation experiments, tumors formed in mice implanted with 100 CD44^{hi} cells in 4/5 and 5/5 mice, respectively, but no tumors formed in mice implanted with an equal number of CD44⁻ cells.

FIGS. 9A-9E are representative micrographs (10×) that show moderate differentiation of CD44^{hi} cells into glandular structures resembling those of the original primary tumor and subsequent primary xenograft tumors from which they were derived (patient sample CT4). FIGS. 9A-9D depict fixed sections stained with hematoxylin and eosin from the CT4 primary tumor (FIG. 9A), subsequent passage 1 (FIG. 9B) and passage 2 (FIG. 9C) xenograft tumors established from the primary tumor, and a tumor derived from 10,000 CT4 passage 2 CD44^{hi} isolated cells (FIG. 9D). FIG. 9E depicts a xenograft passage 2 tumor section stained with periodic acid Schiff (PAS) stain (light gray), which indicates the presence of mucin-secreting goblet cells. PAS stain was performed with diastase treatment to rule out glycogen staining, which is diastase sensitive.

FIGS. 10A-10F show that isolated CD44^{hi} cells form tumors that recapitulate the histology and expression of colon tumor-associated markers found on the original patient tumors from which they were derived (patient sample CT4). Hematoxylin and eosin (H & E) sections (FIGS. 10A-10C, magnification 200×) illustrate the maintenance of moderately differentiated tumor histology and cytokeratin 20 (CK20) staining before (FIG. 10D) and after (FIG. 10E) xenograft passage. FIGS. 10C and 10F show a tumor derived from 100 CD44^{hi} cells isolated from the tumor shown in FIGS. 10B and 10E.

FIGS. 11A-11F show poorly differentiated colon adenocarcinoma (Patient CT3). Tumor tissue sections stained with hematoxylin and eosin (FIGS. 11A-11C, magnification 200×) illustrate the maintenance of poorly differentiated tumor histology and carcinoembryonic antigen (CEA) staining (FIG. 11D-11F) in patient sample CT3 before (FIG. 11A) and after (FIG. 11B) xenograft passage. FIG. 11C shows a tumor derived from a 100 CD44^{hi} cells isolated from the CT3 primary xenograft.

FIGS. 12A-12C show co-expression of CD44^{hi} and stem cell transcription factors. FACS analysis was performed with gating of CD44 APC and subgating of either Oct-3/4 (FIG. 12A) or isotype matched control antibody (FIG. 12B), as described in Example 6.

FIG. 12A shows gating of CD44^{hi} in R1 and CD44⁻ in R2, and subgating of co-expressing CD44^{hi} Oct-3/4⁺ cells in R1b and single positive CD44⁻ Oct-3/4⁻ cells in R2b.

FIG. 12B shows gating of CD44⁻ in R1 and CD44⁻ in R2, and subgating of cells not labeled with control antibody in R1b and R2b.

FIG. 12C is a bar graph showing the percentage of Oct-3/4, Sox-2, and Sox-9 expressing cells that are also CD44^{hi} (black bar) or that lack or show reduced CD44 expression (grey bar). The fold increase in the number of co-expressing CD44^{hi} Oct-3/4⁺ cells as compared to the number of Oct-3/4, Sox-2, and Sox-9 positive cells that don't express CD44 is shown.

FIGS. 13A-13B depict the results of FACS analysis of primary colon tumor xenograft cells from patient CT4 using CD44 and CD166 (FIG. 13A) or CD44 and CD201 (FIG. 13B) for cell selection. CD166 is co-expressed with CD44^{hi} cells (FIG. 13A, gate R7). CD201 is also co-expressed with CD44^{hi} cells (FIG. 13B, gate R7).

FIGS. 14A-14C depict the results of FACS analysis of primary colon tumor xenograft cells from patients CT3 (FIGS. 14A-14B) and CT5 (FIG. 14C) using CD44 and CD166 (FIGS. 14A-14B) or CD44 and CD201 (FIG. 14C) for cell selection. The majority of cells expressing IGF1R and EGFR are also CD44^{hi}.

FIGS. 15A-15D show that CD133⁺ cells have significantly enriched clonogenic growth, and CD117⁺ cells show slightly enriched clonogenic in vitro. CT1 colon tumor cells were sorted by expression of CD133 (FIGS. 15A-15B) or CD117 (FIG. 15C).

FIGS. 15A-15C show in vitro growth of colon tumor cells as assessed in soft agar assays as described in Example 7. Data is shown as the average number of colonies per plate ±SD from at least 2 experiments.

FIG. 15D shows a representative field of soft agar plates from CT1 CD133⁺ sorted cells, showing representative small and large colonies.

### DETAILED DESCRIPTION OF THE INVENTION

The present invention provides methods for the prospective identification of cancer stem cells that express CD44^{hi}, ABCG2, CD133, CD117, and/or ALDH. These cells are highly tumorigenic in vitro and in vivo, are self-renewing, and have the ability to differentiate. The disclosed cancer stem cell populations may also show apoptosis resistance and contribute to cancer relapse and metastasis. Also provided are methods for isolating cancer stem cell populations and for enriching cancer stem cells within a population.

The cancer stem cell populations disclosed herein are useful for studying the effects of therapeutic agents on tumor growth, relapse, and metastasis. Isolated cancer stem cells can be used to identify unique therapeutic targets, which can be used to generate antibodies that target cancer stem cells. The isolated cancer stem cells can also be used in screening assays to improve the probability that drugs selected based upon in vitro activity, or based upon cytotoxicity of tumor populations that include non-tumorigenic cells, will successfully eradicate disease and prevent relapse in vivo. Cancer stem cells isolated from patients may also be used to predict disease outcome and/or sensitivity to known therapies.

### I. Cancer Stem Cells

A stem cell is known in the art to mean a cell (1) that is capable of generating one or more kinds of progeny with reduced proliferative or developmental potential (e.g., differentiated cells); (2) that has extensive proliferative capacity; and (3) that is capable of self-renewal or self-maintenance. See e.g., Potten et al., Development, 1990, 110: 1001-1020. In normal adult animals, some cells (including cells of the blood, gut, breast ductal system, and skin) are constantly replenished from a small population of stem cells in each tissue. Thus, the maintenance of tissues (whether during normal life or in response to injury and disease) depends upon the replenishing of the tissues from precursor cells in response to specific developmental signals.

The best-known example of adult cell renewal by the differentiation of stem cells is the hematopoietic system. Developmentally immature precursors such as hematopoietic stem cells and progenitor cells respond to molecular signals to gradually form the varied blood and lymphoid cell types. Stem cells are also found in other tissues, including epithelial tissues (Slack, Science, 2000, 287: 1431-1433) and mesenchymal tissues (U.S. Pat. No. 5,942,225). Cancer stem cells may arise from any of these cell types, for example, as a result of genetic damage in normal stem cells or by the dysregulated proliferation of stem cells and/or differentiated cells.

Cancer stem cells of the present invention may be derived from any cancer comprising tumorigenic stem cells, i.e., cells having an ability to proliferate extensively or indefinitely, and which give rise to the majority of cancer cells. Within an established tumor, most cells have lost the ability to proliferate extensively and form new tumors, and a small subset of cancer stem cells proliferate to thereby regenerate the cancer stem cells as well as give rise to tumor cells lacking tumorigenic potential. Cancer stem cells may divide asymmetrically and symmetrically and may show variable rates of proliferation. Cancer stem cells of the present invention may also include transit amplifying cells (TACs) or progenitor cells that have reacquired stem cell properties.

Representative cancers from which stem cells may be isolated include cancers characterized by solid tumors, including for example, fibrosarcoma, myxosarcoma, liposarcoma, chondrosarcoma, osteogenic sarcoma, chordoma, angiosarcoma, endotheliosarcoma, lymphangiosarcoma, synovioma, lymphangioendotheliosarcoma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

Additional representative cancers from which stem cells can be isolated or enriched for according to the present invention include hematopoietic malignancies, such as B cell lymphomas and leukemias, including but not limited to low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL and Waldenstrom's Macroglobulinemia, chronic leukocytic leukemia, acute myelogenous leukemia, acute lymphoblastic leukemia, chronic lymphocytic leukemia, chronic myelogenous leukemia, lymphoblastic leukemia, lymphocytic leukemia, monocytic leukemia, myelogenous leukemia, and promyelocytic leukemia.

In contrast to cancer stem cells, non-tumorigenic cancer cells fail to form a palpable tumor upon transplantation into an immunocompromised host, wherein if the same number of non-fractionated, dissociated cancer cells were transplanted under the same circumstances, the cancer stem cells would form a palpable tumor in the same period of time. A palpable tumor is known to those in the medical arts as a tumor that is capable of being handled, touched, or felt.

I.A. Cancer Stem Cell Markers

Cancer stem cells may be selected by positive and negative selection of molecular markers. Cellular surface markers are particularly useful since such markers facilitate in vivo selection. A reagent that binds to a cancer stem cell positive marker (i.e., a marker expressed by cancer stem cells at elevated levels compared to non-tumorigenic or differentiated cells) can be used for the positive selection of cancer stem cells. A reagent that binds to a cancer stem cell negative marker (i.e., a marker not expressed or expressed at measurably reduced levels by cancer stem cells) can be used for the elimination of those cancer cells in the population that are not cancer stem cells. For both positive selection and negative selection, useful markers include those that are expressed on the cell surface such that live cells are amenable to sorting.

Positive markers for cancer stem cells may be present on non-tumorigenic cancer cells, i.e., cancer cells other than cancer stem cells, at reduced or elevated levels. Specifically, a positive marker for cancer stem cells shows positive expression and a measurable difference in level of expression as compared to non-tumorigenic cancer cells. When a positive marker for cancer stem cells shows positive but reduced expression when compared to non-tumorigenic cancer cells, high level expression of the same marker can also be used for negative selection.

For example, CD44 is expressed on the majority of colon cancer cells, which initially suggested that CD44 was not a useful marker for isolating a cancer stem cell subfraction of colon tumor cells. However, markers that are widely expressed may be show a measurable change in expression level in cancer stem cells and/or may provide for resolution of cancer stem cells when used in combination with additional positive or negative markers. Representative positive cancer stem cell markers include CD44^{hi}, ABCG2, β-catenin, CD117, CD133, ALDH, VLA-2, CD166, CD201, IGFR, EGF1R, Tweak (TNF-like weak inducer of apoptosis), EphB2, EphB3, human Sca-1 (BIG1), CD34, ESA, β1 integrin (CD29), CD90, CD150, and CXCR4, among others known in the art. Cancer stem cell markers are typically expressed at a level that is at least about 5-fold greater than differentiated cells of the same origin or non-tumorigenic cells, for example, at least about 10-fold greater, or at least about 15-fold greater, or at least about 20-fold greater, or at least about 50-fold greater, or at least about 100-fold greater.

Representative negative cancer stem cell markers include molecules expressed in differentiated cancer cells of the same origin or in non-tumorigenic cells. For example, as goblet, absorptive, and endocrine cells of the mature colon, may be identified with cell surface or cytoplasmic markers such as Muc-1, CD26, and chromagranin A, respectively. Goblet cells also express Muc-2 and show positive staining with periodic acid Schiff (PAS). Differentiated absorptive cells express villin.

Disclosed herein are CD44^{hi}, ABCG2, β-catenin, CD117, CD133, ALDH, VLA-2, CD166, CD201, IGFR, and/or EGF1R markers that can be used alone or in combination for the prospective identification and isolation of cancer stem cells from colon. CD44 is a transmembrane glycoprotein that participates in cancer metastasis by modulating cell adhesiveness, motility, matrix degradation, proliferation, and/or cell survival. See e.g., Marhaba & Zoller, J. Mol. Histol., 2004, 35(3): 211-231. ABCG2 is the receptor responsible for the side population (SP) phenotype of cells found to have cancer stem-like properties in prostate and brain cancer (Patrawala et al., Cancer Res., 2005, 65(14): 6207-6219; Kondo et al., Proc. Natl. Acad. Sci. U.S.A., 2004, 101(3): 781-786). ABCG2 has also been identified as a marker of cancer stem cells in acute myeloid leukemia (Wulf et al., Blood, 2001, 98(4): 1166-1173). CD133 and CD117 have been described as markers for hematopoietic stem cell populations. CD26 is a cell surface glycoprotein marker of differentiation that is used for negative selection, i.e., isolated or enriched cancer stem cell population lack or are depleted of cells expressing CD26. Markers used for negative selection of cancer stem cells show a level of expression in cancer stem cells that is at least about 5-fold less than a level of expression in differentiated cells or normal non-tumorigenic cell types, for example, at least about 10-fold less, or at least about 15-fold less, or at least about 20-fold less, or about 50-fold less, or about 100-fold less.

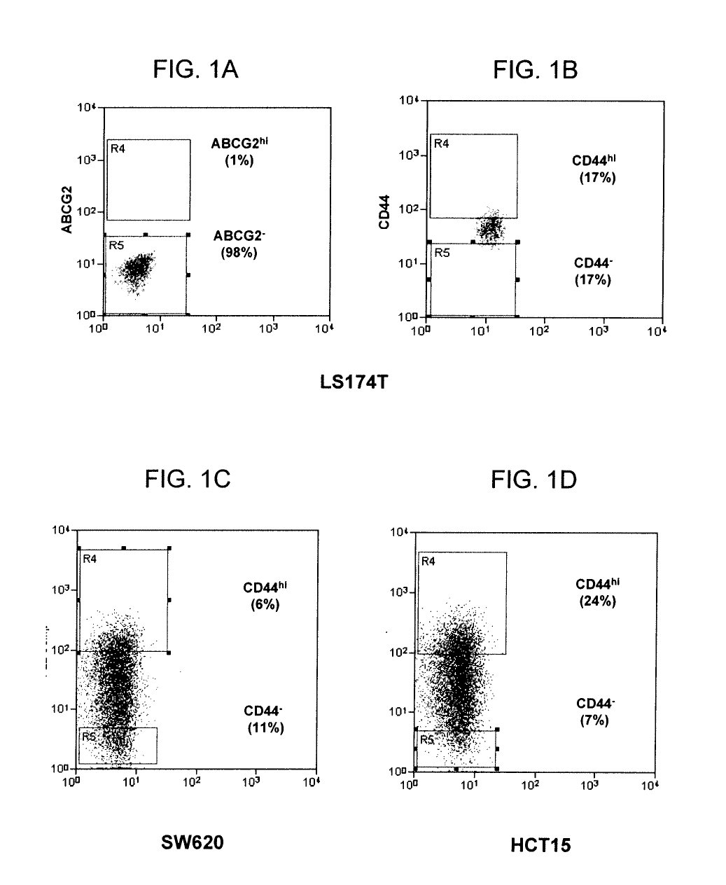

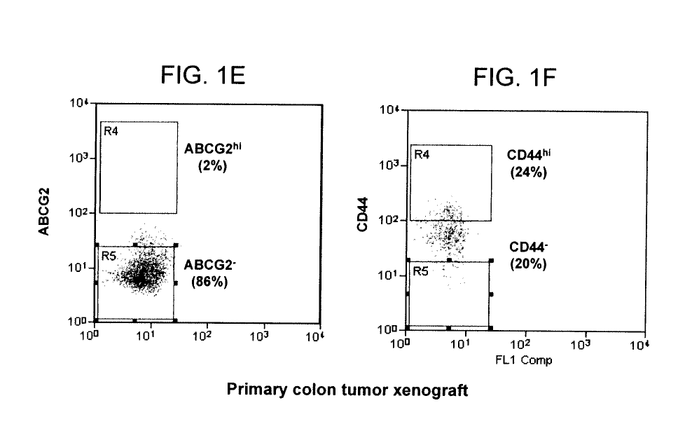

As described in Example 2, CD44 was expressed on all colon tumor cells and primary tumors tested, whereas ABCG2 was expressed on about 67% of samples (6/9). Isolation of CD44 cells having the highest levels of expression (CD44^{hi}) resulted in purification of about 20-30% of the tumor cells. When present, ABCG2 was expressed on a very small subset (less than approximately 2.0%) of colon tumor cells. See also Table 1 and FIGS. 1A-1E.

In a particular aspect of the invention, an isolated cancer stem cell population comprise at least 90% cancer stem cells, wherein the cancer stem cells express CD44^{hi}, ABCG2, β-catenin, CD117, CD133, ALDH, VLA-2, CD166, CD201, IGFR, and/or EGF1R at a level that is at least about 5-fold greater than CD44⁻ non-tumorigenic cells of the same origin. Cancer stem cells may also express ABCG2 or express CD44 at a level that is at least about 10-fold greater than CD44⁻ non-tumorigenic cells of the same origin, for example, at least about 15-fold greater, or at least about 20-fold greater, or at least about 50-fold greater, or at least about 100-fold greater. An isolated cancer stem cell population is removed from its natural environment (such as in a solid tumor) and is at least about 75% free of other cells with which it is naturally present and which lack or show measurably reduced levels of the marker based on which the cancer stem cells were isolated. For example, isolated cancer stem cell populations as disclosed herein are at least about 90%, or at least about 95%, free of non-tumorigenic cells. When referring to a cancer stem cell population that is described as a percentage purity, or a percentage free of non-tumorigenic cells, the cell stem cell subpopulation and total cancer cell population are typically quantified as live cells.

In another aspect of the invention, an enriched cancer stem cell population isolated from a tumor cell population comprises cancer stem cells and non-tumorigenic cells, wherein the cancer stem cells express ABCG2 or express CD44 at a level that is at least about 5-fold greater than non-tumorigenic cells of the same origin, or at least about 10-fold greater, or at least about 15-fold greater, or at least about 20-fold greater, or at least about 50-fold greater, or at least about 100-fold greater. An enriched population of cells can be defined based upon the increased number of cells having a particular marker in a fractionated cancer stem cell population as compared with the number of cells having the marker in the non-fractionated cancer cell population. It may also be defined based upon tumorigenic function as the minimum number of cells that form tumors at limiting dilution frequency. An enriched cancer stem cell population can be enriched about 2-fold in the number of stem cells as compared to the non-fractioned tumor cell population, or enriched about 5-fold or more, such as enriched about 10-fold or more, or enriched about 25-fold or more, or enriched about 50-fold or more, or enriched about 100-fold or more. Enrichment can be measured with using any one of the cancer stem cell properties noted herein above, e.g., levels of marker expression or tumorigenicity.

The present invention provides methods for isolation of the disclosed cancer stem cell populations. For example, the method can comprise (a) providing dissociated tumor cells, wherein a majority of the cells either do not express CD44 or express CD44 at a low level, and wherein a minority of the cells express CD44 at a high level that is at least about 5-fold greater than the low level; (b) contacting the dissociated tumor cells with an agent that specifically binds to CD44; (c) selecting cells that specifically bind to the agent of (b) to an extent that shows a high level of CD44 expression that is at least about 5-fold greater than the low level. The method can also comprise (a) providing dissociated tumor cells; (b) contacting the dissociated tumor cells with an agent that specifically binds to ABCG2; (c) selecting cells that specifically bind to the agent of (b) at a level that is at least about 5-fold greater than cells that either do no express ABCG2 or express ABCG2 at a low level. Representative methods for isolation of ABCG2^{hi} and/or CD44^{hi} cancer stem cell populations are described in Examples 1-2.

The method can further comprise selecting cancer stem cells using one or more of the additional positive stem cell markers as noted above (e.g., CD117, CD133, ALDH, VLA-2, β-catenin, VLA-2, CD166, CD201, IGFR, EGF1R, Tweak (TNF-like weak inducer of apoptosis), EphB2, EphB3, human Sca-1 (BIG1), CD34, ESA, β1 integrin (CD29), CD90, CD150, and CXCR4, IGF1-R, GPR49, CD166, and/or CD201, among others known in the art), either alone or in combination with CD44 and/or ABCG2. For example, CD44^{hi} cells also coexpress VLA-2 (a receptor for ADAM9-S), β-catenin, CD117, CD133, ALDH, CD166, CD201, IGFR, EGF1R, and proteins encoded by the genes identified in Table 8. See Example 6. As another example, GPR49 is co-expressed with CD44. See also Dalerba et al., Proc. Natl. Acad. Sci. U.S.A., 2007 104(24):10158-10163. When selecting cells that express high levels of CD44 or ABCG2, or cells that express additional positive stem cell markers, cancer stem cells may be identified as cells that show a level of expression of the marker that is at least about 5-fold greater than a baseline level (i.e., a background level of staining due to non-specific binding or low levels of binding), or at least about 10-fold greater than a baseline level, or at least about 15-fold greater than a baseline level, or at least about 20-fold greater than a baseline level, or at least about 50-fold greater, or at least about 100-fold greater. Cancer stem cells selected using the disclosed markers show increased tumorigenic potential and other cancer stem cell properties described herein, such as increased clonogenicity, self-renewal, and an ability to generate tumors with differentiated cells. For example, CD33⁺ and CD117⁺ cells also show tumorigenic properties of stem cells. See Example 7.

The disclosed methods can also include a negative step selection, e.g., excluding cells that express one or more markers expressed in differentiated cells of the same tissue type, or excluding cells that show reduced levels of expression of a particular marker. For example, cancer stem cells from colon show reduced expression of the differentiation marker CD26. See Example 6. Additional representative differentiation markers for colon include CD24, Muc-1, Muc-2, and villin, among others known in the art. Negative markers can also include antigens associated with normal cell types and which are undetectable or show similarly reduced expression in cancer stem cells. See e.g., Table 8, genes downregulated in CD44^{hi} cells as compared to CD44⁻ cells. When selecting cells that show low or undetectable levels of CD26 or other negative stem cell markers, cancer stem cells may be identified as cells that show a level of expression of the marker that is at least about 5-fold less in cancer stem cells as compared to differentiated cells or normal cell types, or at least about 10-fold less, or at least about 15-fold less, or at least about 20-fold less, or about 50-fold less, or about 100-fold less.

Cancer stem cells can be isolated by any suitable means known in the art, including FACS using a fluorochrome conjugated marker-binding reagent. Any other suitable method including attachment to and disattachment from solid phase, is also within the scope of the invention. Procedures for separation may include magnetic separation, using antibody-coated magnetic beads, affinity chromatography and panning with antibody attached to a solid matrix, e.g., a plate or other convenient support. Techniques providing accurate separation include fluorescence activated cell sorters, which can have varying degrees of sophistication, such as multiple color channels, low angle and obtuse light scattering detecting channels, impedance channels, etc. Dead cells may be eliminated by selection with dyes that bind dead cells (such as propidium iodide (PI), or 7-AAD). Any technique may be employed that is not unduly detrimental to the viability of the selected cells.

I.B. Enriched Clonogenicity of Cancer Stem Cells

As described herein above, cancer stem cells of the invention are tumorigenic in vitro and in vivo, have characteristics of tumorigenic cells such as clonogenicity, and a highly proliferative nature. Subpopulations of colon tumor cell lines were identified that express ABCG2^{hi} and CD44^{hi} and that are significantly enriched for in vitro soft agar colony formation and proliferation. ABCG2^{hi} and CD44^{hi} cells isolated from a primary tumor xenograft established from a fresh colon tumor sample were also enriched for soft agar colony formation and showed improved viability. See Example 3.

In vivo proliferation of cancer stem cells can be accomplished by injection of cancer stem cells into animals, such as mammals, particularly mammals used as laboratory models. For example, cancer stem cells may be injected into immunocompromised mice, such as SCID mice, Beige/SCID mice, or NOD/SCID mice. NOD/SCID mice are injected with varying number of cells and observed for tumor formation. The injection can be by any method known in the art, following the enrichment of the injected population of cells for cancer stem cells. The injection of cancer stem cells can consistently result in the successful establishment of tumors more than 75% of the time, such as more than 80% of the time, or more than 85%, or more than 90%, or more than 95% of the time, or 100% of the time.

As described in Example 4, in vivo tumorigenicity experiments were performed by subcutaneous implantation of sorted cells from four primary tumor xenografts into immunodeficient mice at cell numbers titrated in 10-fold increments from 1,000,000 down to 10 cells. CD44^{hi} and ABCG2^{hi} cells were at least about 10-fold more tumorigenic than CD44⁻ and ABCG2⁻ cells, respectively, generating tumors with fewer numbers of cells, and with significantly shorter latency, more aggressive growth, and larger mean tumor volume. As few as 10 CD44^{hi} cells formed tumors in 7/10 mice, and 100 ABCG2^{hi} cells formed tumors in 5/9 mice, whereas 0 and 1 tumors formed in matched CD44⁻ and ABCG2⁻ control groups, respectively, monitored for up to 6 months. Expression of ALDH and reduced expression of CD26 also correlated with increased tumorigenicity.

I.C. Capacity of Cancer Stem Cells to Differentiate

Cancer stem cells of the invention give rise to tumors with the same differentiation state of the tumor of origin. For example, cancer stem cells isolated from poorly and moderately differentiated tumors give rise to poorly and moderately differentiated tumors in vivo, respectively. The molecular profile of the resultant tumors are also similar to the tumor of origin, notwithstanding the prior selection of cancer stem cells. Thus, the cancer stem cells show a capacity to differentiate or give rise to non-tumorigenic cells that make up the majority of mature cancer populations.

Isolated CD44^{hi} and ABCG2^{hi} colon tumor cells generated tumors with both CD44^{hi} and CD44⁻, or ABCG2^{hi} and ABCG2⁻ cells, respectively. See Example 5. In addition, the approximate ratio of CD44^{hi} to CD44⁻ cells in the parental tumor xenografts was also observed in secondary tumors whether 10, 100, or 1,000 CD44^{hi} cells were used to generate the tumor. Similarly, isolated ABCG2^{hi} cells, which represented only about 2% of the parental tumor population, also gave rise to tumors that had approximately 2% of ABCG2^{hi} cells. Resultant tumors also expressed differentiation markers such as CEA, CK20, CD26, Muc-1, and mucin. Thus, CD44^{hi} and ABCG2^{hi} cells retain an innate ability to give rise to daughter cells with a mixed but defined pattern of CD44 and ABCG2 expression, which indicates a capacity for differentiation.

I.D. Self-Renewal of Cancer Stem Cells

The cancer stem cells of the invention have a capacity for self-renewal, as demonstrated by the ability of CD44^{hi} but not CD44⁻ cells to consistently form tumors with as few as 100 implanted cells in 4 rounds of serial transplantations. While the cancer stem cells may be capable of symmetric and asymmetric mitosis, the capacity for self renewal is based upon an ability to undergo asymmetric cell divisions. This feature allows cancer stem cells to retain multipotency and high proliferative potential throughout repeated cell divisions. See Example 5.

### II. Applications

The cancer stem cell populations disclosed herein are useful for studying the effects of therapeutic agents on tumor growth, relapse, and metastasis. When isolated from a cancer patient, the efficacy of particular therapies can be tested and/or predicted based upon the unique genetic and molecular profile of the isolated population. Thus, the disclosed cancer stem cell populations provide means for developing personalized cancer therapies.

In one aspect of the invention, the genetic and molecular features of cancer stem cells are described to identify target molecules and/or signaling pathways. Accordingly, the present invention also provides arrays or microarrays containing a solid phase, e.g., a surface, to which are bound, either directly or indirectly, cancer stem cells (enriched populations of or isolated), polynucleotides extracted from cancer stem cells, or proteins extracted from the cancer stem cells. Monoclonal and polyclonal antibodies that are raised against the disclosed cancer stem cell populations may be generated using standard techniques. The identification of cancer stem cell target molecules, and agents that specifically bind cancer stem cells, will complement and improve current strategies that target the majority non-tumorigenic cells.

Microarrays of genomic DNA from cancer stem cells can also be probed for single nucleotide polymorphisms (SNP) to localize the sites of genetic mutations that cause cells to become precancerous or tumorigenic. The genetic and/or molecular profile of cancer stem cells may also be used in patient prognosis. See e.g., Glinsky et al., J. Clin. Invest., 2005, 115(6): 1503-1521, which describes a death-from-cancer signature predicting therapy failure.

In another aspect of the invention, the efficacy of cancer drugs or candidate cancer drugs can be tested by contacting isolated cancer stem cells with a test compound and then assaying for a change in cancer stem cell properties as described herein. For example, therapeutic compositions can be applied to cancer stem cells in culture at varying dosages, and the response of these cells is monitored for various time periods. Physical characteristics of these cells can be analyzed by observing cells by microscopy. Induced or otherwise altered expression of nucleic acids and proteins can be assessed as is known in the art, for example, using hybridization techniques and Polymerase Chain Reaction (PCR) amplification to assay levels of nucleic acids, immunohistochemistry, enzymatic assays, receptor binding assays, enzyme-linked immunosorbant assays (ELISA), electrophoretic analysis, analysis with high performance liquid chromatography (HPLC), Western blots, radioimmunoassays (RIA), fluorescence activated cell sorting (FACs), etc.

The ability of therapeutic compounds to inhibit or decrease the tumorigenic potential of cancer stem cells can be tested by contacting cancer stem cells and a test compound, allowing a sufficient temporal period for response, and then assessing cancer stem cell growth in vitro, for example, using soft agar assays as described in Example 3. Following exposure to the test compound, the cancer stem cells can alternatively be transplanted into a host animal (i.e., preparation of a xenograft model as described in Example 4), which is then monitored for tumor growth, cancer cell apoptosis, animal survival, etc. In yet another screening format, test compounds are administered to a xenograft host animal (i.e., an animal bearing cancer stem cells and/or a resultant tumor). Additional phenotypes that may be assayed include cell viability, proliferation rate, regenerative capacity, and cell cycle distribution of cancer stem cells or resultant non-tumorigenic cancer cells, or any other phenotype relevant to therapeutic outcome.

Test compounds include known drugs and candidate drugs, for example, viruses, proteins, peptides, amino acids, lipids, carbohydrates, nucleic acids, antibodies, prodrugs, small molecules (e.g., chemical compounds), or any other substance that may have an effect on tumor cells whether such effect is harmful, beneficial, or otherwise. Test compounds can be added to the culture medium or injected into the mouse at a final concentration in the range of about 10 pg/ml to 1 μg/ml, such as about 1 ng/ml (or 1 ng/cc of blood) to 100 ng/ml (or 100 ng/cc of blood).

For use in any of the above-noted applications, or other applications, cancer stem cells of the invention may be cryopreserved until needed for use. For example, the cells can be suspended in an isotonic solution, preferably a cell culture medium, containing a particular cryopreservant. Such cryopreservants include dimethyl sulfoxide (DMSO), glycerol and the like. These cryopreservants are used at a concentration of 5-15%, such as 8-10%. Cells are frozen gradually to a temperature of −10° C. to −150° C., such as −20° C. to −100° C., or at −150° C.

### EXAMPLES

The following examples have been included to illustrate modes of the invention. Certain aspects of the following examples are described in terms of techniques and procedures found or contemplated by the present co-inventors to work well in the practice of the invention. In light of the present disclosure and the general level of skill in the art, those of skill will appreciate that the following examples are intended to be exemplary only and that numerous changes, modifications, and alterations may be employed without departing from the scope of the invention.

### Example 1

### Flow Cytometry Analysis of Colon Tumor Cells

Colon tumor cell lines LS174T, HT29, HCT15, HCT116, and SW620 were obtained from the American Type Culture Collection (ATCC) and cultured according to ATCC instructions. The cell line CT1 was established from a primary colon adenocarcinoma sample by dissociating fresh tumor with collagenase and DNAse I, and then culturing tumor cells in RPMI supplemented with 10% fetal bovine serum (FBS), 20 ng/mL of epidermal growth factor (BD Biosciences of San Jose, Calif.), basic fibroblast growth factor (BD Biosciences), leukemia inhibitory factor (Chemicon of San Diego, Calif.), stem cell factor (Stem cell technologies of Vancouver, Canada), L-glutamine, 1 μg/mL hydrocortisone, 4 μg/mL hydrocortisone, 5 μg/mL insulin, and penicillin/streptomycin.

Primary colon adenocarcinoma tumor samples were obtained from patients undergoing surgical resection at Grossmont Hospital (San Diego, Calif.) and used to establish primary human xenograft tumors in immune deficient mice. All mice were obtained from Charles River Laboratory (Wilmington, Mass.) and maintained under pathogen-free conditions according to IACUC guidelines. Xenograft tumors (passage 1) were established by implanting 1-3 mm³ tumor fragments into the kidney capsule of NOD/Scid mice or subcutaneously into the right flank of female Scid/Bg mice. All subsequent passages were by subcutaneous implant of female Scid/Bg mice. Hematoxilin and eosin stain of fixed sections from tumor xenografts were similar in histologic grade to original primary tumors, and stained positive for human epithelial markers (AE1/AE3 and EPCAM), and human colon tumor markers (CEA and cytokeratin 20). Characteristics of the primary tumors used in these experiments are shown in Tables 1-2.

To prepare single cell suspensions of tumor tissue for in vitro and in vivo assays, tumors from 4-6 animals were rinsed 4-5 times in RPMI-1640 medium supplemented with gentamicin (50 μg/mL) and FUNGIZONE® (0.25 μg/mL), debrided of necrotic tissue, and then minced using sterile razor blades in a glass dish. All steps were performed aseptically. Minced tissues were digested in 0.1% collagenase type IV (Sigma-Aldrich of St. Louis, Mo.) and 0.01% DNAse I (Sigma-Aldrich of St. Louis, Mo.) in RPMI-1640 for 15-20 minutes with constant stirring at room temperature. The digested material was pipetted to break up clumps and filtered through a tissue disaggregation screen. Cells were then washed 2 times, counted, and filtered again through a 40 μM or 70 μM nylon mesh screen prior to flow cytometric analysis and sorting.

Cells were stained for flow cytometry at 4° C. for 20-25 minutes in RPMI-1640 containing 3% FBS using the following monoclonal antibodies (all from BD Pharmingen of San Diego, Calif., unless otherwise noted): anti-Ms H2Dd and anti-Ms H2 Kd mAb, anti-CD44 mAb, anti-CD26, anti-CD117, anti-ABCG2 (Chemicon), and anti-CD133-PE (Miltenyi Biotec of Auburn, Calif.). Non-epithelial cells from fresh, unpassaged human tumors were excluded by staining with antibodies to CD2, CD3, CD10, CD16, CD18, CD31, CD64, and CD140b, essentially as described by Al-Hajj et al., Proc. Natl. Acad. Sci. USA, 2003, 100(7): 3983-3988. All antibodies were directly conjugated to fluorescein isothiocyanate (FITC), phycoerythrin (PE), or phycocyanin alophycocyanin, phycoerythrin-cyanin dye 5 (PeCy5). After staining, cells were washed 2 times, and resuspended in RPMI-3% FBS containing 1 μg/mL propidium iodide (PI) prior to flow cytometric analysis on a MOFLO® cell sorter (Dako Colorado, Inc. of Fort Collins, Colo.). Cell debris and doublets or aggregates were excluded by forward and side scatter, and pulse-width gating, respectively. Mouse lineage cells present in xenograft tumor preparations were excluded by positive staining with anti-Ms H2Dd/anti-Ms H2 Kd. Cells were sorted to greater than about 90% purity, as determined by subjecting sorted cells to a second FACS analysis.

### Example 2

### Colon Cancer Cells Contain Subpopulations of CD44^{hi} and ABCG2^{hi} Cells

The expression of CD44 and ABCG2 was studied in colon tumor cells using FACS cell sorting as described in Example 1. ABCG2 expression was employed as a surrogate for side population (SP) cells because of potential toxicities and related complications in data interpretation resulting from staining cells with Hoescht 33342.

In a panel of colon tumor cell lines, the percentage of ABCG2^{hi} cells was low, between 0-2%. See FIG. 1A and Table 1. Similar data was obtained using fresh primary colon tumor samples or primary tumor xenografts established from fresh colon tumor samples. See FIG. 1E and Table 1. Primary (1°) tumor xenografts (CT2, CT3, CT4, and CT5) were established by in vivo passage of primary colon adenocarcinoma surgical samples obtained from each of four patients and included poorly and moderately differentiated tumors. The tumors were passaged 1 to 4 times with no significant differences noted in CD44 or ABCG2 expression between the passages. Three of five (3/5) patient samples tested had a small subpopulation of brightly staining ABCG2^{hi} cells (range=0.3-2.0%).

TABLE 1
Expression of CD44 and ABCG2 on Colon Cancer Cells

| | CD44^{hi} | | |
|---|---|---|---|
| Cell lines | Assay 1 | Assay 2 | ABCG2^{hi} |
| LS174T | 17 | ND | 1 |
| HT29 | 95 | ND | 0.7 |
| HCT15 | 16 | ND | 0.2 |
| HCT116 | 75 | ND | ND |
| SW620 | 50 | ND | 0.05 |
| CT1 | 21 | 31 | ND |

Passaged Primary Tumors

| | | | |
|---|---|---|---|
| CT2 (poorly differentiated) | 33 | ND | 2 |
| CT3 (poorly differentiated) | 24 | ND | 0 |
| CT4 (moderately differentiated) | 40 | 16 | 0 |
| CT17 (moderately differentiated) | 20 | ND | ND |
| CT5 (moderately differentiated) | 45 | 24 | 0.7 |

Unpassaged Primary Tumors

| | | | |
|---|---|---|---|
| CT8-u (infiltrating) | 22/36* | ND | ND |
| CT9-u (well differentiated) | 16/47* | ND | ND |
| CT10-u (moderately differentiated) | 18 | ND | ND |
| CT11-u (moderately differentiated) | 17 | ND | ND |
| CT4-u (moderately differentiated) | 18 | 15 | ND |
| CT17-u (moderately differentiated) | 17 | ND | 1.2 |
| CT5-u (moderately differentiated) | 11 | 10 | 1.5 |

Assay 1 and assay 2 are independent experiments performed in a same manner.
ND = not determined;
CT = colon tumor;
*CD44⁺ values reported for CT8 and CT9

TABLE 2
Expression of CD44 in Colon Cancer Patient Samples

| | | TNM | | % CD44^{hi} | | % CD44^{hi} in |
|---|---|---|---|---|---|---|
| Patient | Tumor Site | Stage | Differentiation | in tumor | Xenograft | xenograft |
| CT1 | Sigmoid | T3 N2 M1 | Poor | | | 31* |
| CT2 | Right | T3 N2 Mx | Poor/moderate | | Yes | 33 |
| CT3 | Right | T4 N1 Mx | Poor | | Yes | 24 |
| CT4 | Left | T3 N0 Mx | Moderate | 15 | Yes | 16 |
| CT5 | Sigmoid | T3 N0 Mx | Moderate | 10 | Yes | 24 |
| CT7 | Right | T4 N0 Mx | Moderate | 24 | | |
| CT8 | Right | T3 N1 Mx | Poor | 22 | | |
| CT9 | Right | T2 N0 Mx | Well | 16 | | |
| CT10 | Sigmoid | T3 N1 Mx | Moderate | 18 | No | |
| CT11 | Right | T3 N1 Mx | Moderate | 17 | Yes | 20 |
| CT12 | Not | T3 N1 Mx | Well | 5 | | |
| | Specified | | | | | |
| CT13 | Rectum | T3 N0 Mx | Well | 5 | | |

Expression of CD44^{hi} determined by FACS (fluorescence-activated cell sorter) analysis is shown for freshly isolated tumor samples (% CD44^{hi} in tumor) and primary tumor xenografts established from patient samples CT2-5 and CT11 (% CD44^{hi} in tumor).
TNM cancer staging system:
T = stage (0-4) based on tumor size and invasiveness,
N = extent of nodal involvement (lymph nodes), and
M = extent of spread (metastasis).
Mx = unable to evaluate.

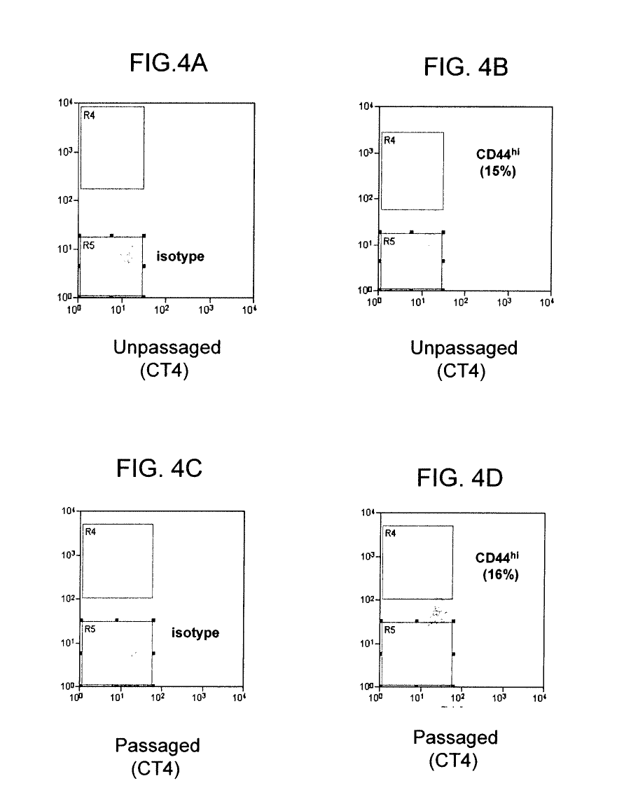

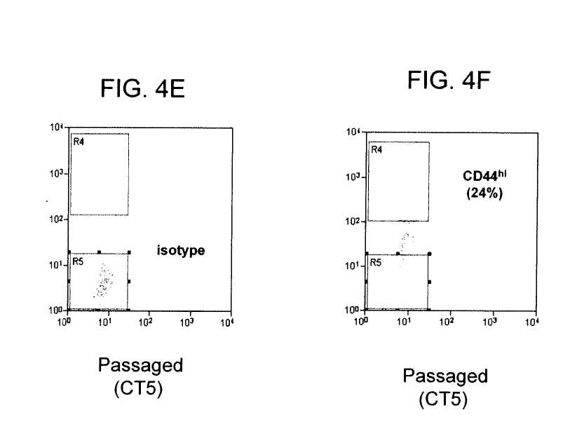

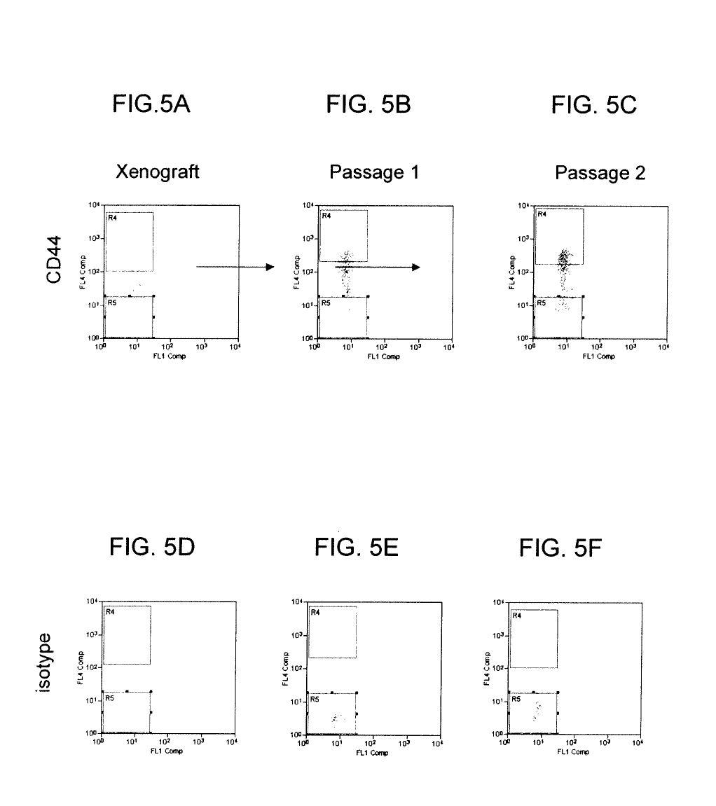

In contrast to ABCG2, CD44 was expressed on the majority of cells in most cell lines tested. This finding at first suggested that CD44 was not an ideal marker for identifying a cancer stem cell subfraction in colon tumor cells. However, it was discovered that most cell lines had a broad pattern of distribution that included a subpopulation of brightly staining cells, which were designated CD44^{hi} cells (Table 1). For example, LS174T cells had 17% CD44^{hi} cells, which were defined by gating on the subfraction of CD44 positive cells that had a fluorescence intensity of approximately one-half (½) log higher than isotype control labeled, or CD44⁻ cells. See FIG. 1B. Primary colon tumor cells, both fresh or xenograft passaged, also had a broad distribution of CD44 expression which allowed for the distinction of CD44^{hi} cells (FIGS. 1F and 4A-4F and Tables 1-2). In most primary colon tumors, there was a distinct, brightly stained CD44^{hi} population with a mean fluorescence intensity (MFI) at least ½ log higher than that of the CD44⁻ tumor population. In some tumors, there was a more continuous distribution of CD44 expression and for these samples, the CD44^{hi} gate was set on cells with an MFI of at least ½ log higher than that of the CD44⁻ population. The average number of CD44^{hi} cells in primary colon tumors was 19%±12 (mean±standard deviation, n=12), and ranged from 5-33% (Table 2). In three samples (CT4, CT5, and CT11) that were analyzed for CD44 expression both before and after xenograft passage, the number of CD44^{hi} cells did not change significantly after xenograft passage. See FIGS. 5A-5F and Table 2).

### Example 3

### CD44^{hi} and ABCG2^{hi} Colon Tumor Cells Are Enriched for In Vitro Proliferation, Clonogenic Growth, and Viability

Colon tumor cells subfractionated based upon expression of CD44 and ABCG2 expression were sorted as described in Example 1 and then seeded in soft agar and/or 96 well plates. For soft agar assays, a bottom layer of 0.6% agar noble (Sigma-Aldrich) in RPMI-1640 (Sigma-Aldrich)+10% FBS was first placed onto 35 mm petri dishes (Stem Cell Technologies of Vancouver, Canada). Tumor cells were seeded at between 5-20,000 cells per dish in warm 0.3% top agar containing RPMI+10% FBS. After 24 hours, dishes were checked to verify that cells were in a single cell suspension. Fresh top agar was added after 10 days, and colonies were counted between 10-28 days using an inverted light microscope (Zeiss of Thornwood, N.Y.). For cell viability/proliferation assays, 5,000 cells were seeded in triplicate in 96-well plates for 48 hours, then assayed using CELLTITER-GLO®, a luminescence based ATP assay, according to the manufacturer's instructions (Promega of Madison, Wis.).

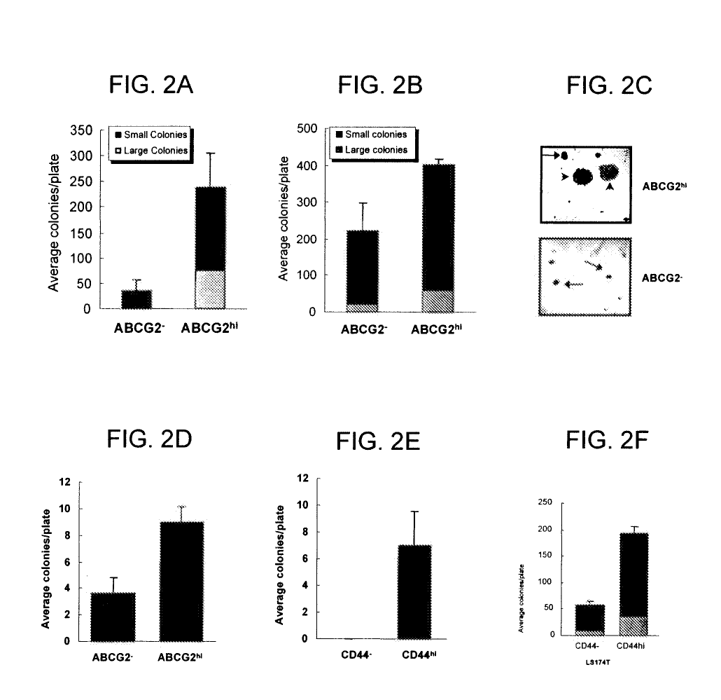

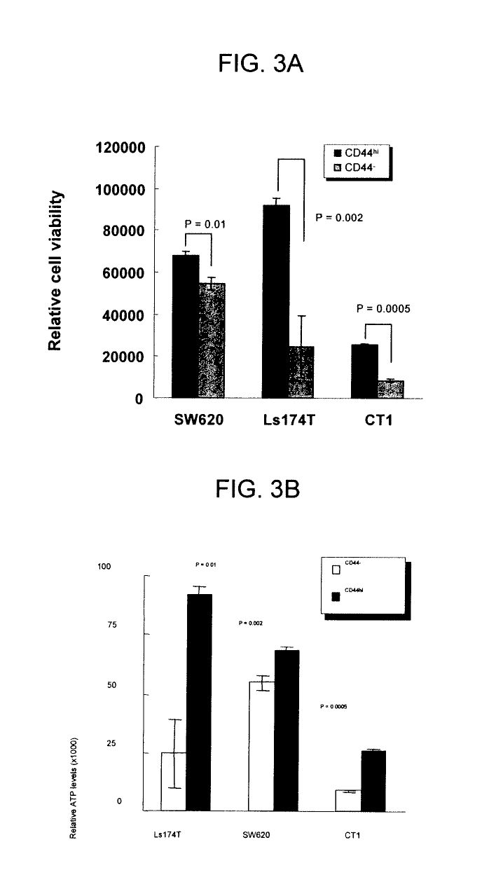

ABCG2^{hi} cells sorted from LS174T and HT29 cells formed significantly more colonies than matched ABCG2⁻ cells, as shown in FIGS. 2A-2B. ABCG2^{hi} derived colonies were also bigger than colonies derived from ABCG2⁻ cells (FIGS. 2A-2C), or parental unsorted cells. CD44^{hi} cells sorted from LS174T also formed significantly more colonies than matched CD44⁻ cells (FIG. 2F). Small (20-100 cells) and large (>100 cells) from LS174T were counted separately, as indicated by grey and black bars in FIG. 2F. CD44^{hi} cells sorted from LS174T, SW620, and a low passage primary colon tumor cell line CT1 also had significantly higher levels of ATP than matched CD44⁻ cells, indicating that CD44^{hi} cells had improved viability than matched CD44⁻ cells (FIGS. 3A-B).

Sufficient quantities of sorted cells from fresh surgical tumor samples were difficult to obtain, and therefore, fresh tumor samples were expanded by passaging them in vivo in immune deficient mice. Primary colon tumor xenografts were established using fresh tumor samples from two poorly differentiated colon adenocarcinomas, CT2 and CT3. Cells sorted from primary xenografts formed relatively few colonies in soft agar, which is not unexpected given that these cells were derived from in vivo passage and were not adapted for in vitro growth. However, ABCG2^{hi} primary tumor xenograft cells from CT2 formed approximately 2.5-fold more colonies in soft agar compared to matched ABCG2⁻ cells (FIG. 2D), consistent with the observation that ABCG2^{hi} cells from tumor cell lines were enriched for soft agar growth ability. CD44^{hi} primary xenograft tumors were also enriched for soft agar growth compared to matched CD44⁻ cells (FIG. 2E). Collectively, these data indicated that CD44^{hi} and ABCG2^{hi} cells were enriched for in vitro clonogenic potential and increased proliferative activity.

CD44^{hi} cells also showed increased viability when compared to CD44⁻ cells (FIGS. 3A-B). Isolated CD44^{hi} and CD44⁻ cells from LS174, SW620, and the primary colon tumor cell line CT1 were analyzed in a cell viability assay by seeding isolated cells in 96-well plates and measuring ATP levels at 48 hours. CD44^{hi} cells were found to have significantly higher levels of ATP, as indicated by p values <0.002 (student's t test). Error bars represent standard error of triplicate samples. This data is representative of two experiments.

### Example 4

### CD44^{hi} and ABCG2^{hi} Primary Colon Tumor Cells Are Highly Tumorigenic In Vivo

Primary colon tumors from each of 5 patients (CT2-5 and CT11) were used to generate tumor xenografts and harvested for tumorigenicity experiments following 2 to 3 passages. Dissociated xenograft tumors were sorted by expression of CD44, depleted of mouse lineage cells using anti-H2D^{d} and H2K^{d} monoclonal antibodies, and injected into the right flank of immune deficient (Scid/Bg) mice. The number of cells injected per animal in initial experiments was titrated in 10-fold dilutions from 1,000,000 to 10 cells. The highest cell implant group for CT5 was 50,000. Cells to be implanted were resuspended in PBS, mixed in an 1:1 ratio with MATRIGEL® (BD Pharmingen of San Diego, Calif.), and a 200 μL final volume injected into the right flank of female Scid/Bg mice.

Tumor development was monitored 1-2 times per week and tumor volume was calculated using the formula (length×width²)/2. Mice were monitored for up to six months until animals had to be euthanized due to obvious tumor burden or illness. Data was recorded as the frequency of mice with palpable tumors in each implantation group by 6 months post-implant. See Table 3. Resultant tumors were removed for further flow cytometry analysis. Tumors were removed and prepared into single cell suspensions for additional flow cytometry and self-renewal analysis essentially as described by Al-Hajj et al., Proc. Natl. Acad. Sci. USA, 2003, 100(7): 3983-3988. Results are presented in Table 3 and are described further below.

TABLE 3
In Vivo Tumorigenicity

| | Xenograft | | | Tumor | Tumor |
|---|---|---|---|---|---|
| Patient | Passage | Cell Type | No. of Cells | Frequency | Latency |
| CT2 | P4 | CD44^{hi} | 100,000 | 9/9 | 19 |
| | | | 10,000 | 9/9 | 23* |
| | | | 1,000 | 9/9 | 34 |
| | | | 100 | 2/5 | 31 |
| | | CD44⁻ | 100,000 | 4/5 | 24 |
| | | | 10,000 | 9/9 | 33 |
| | | | 1,000 | 4/9 | 34 |
| | | | 100 | 0/5 | |
| | | PI⁻ | 1,000,000 | 6/6 | 23 |
| | | | 100,000 | 5/5 | 21 |
| | | | 10,000 | 6/9 | 28 |
| | | | 1,000 | 3/9 | 40 |
| | | | 100 | 0/5 | |
| | | ABCG2^{hi} | 100,000 | 2/2 | 17 |
| | | | 10,000 | 8/8 | 23* |
| | | | 1,000 | 8/9 | 29 |
| | | | 100 | 5/9 | 29 |
| | | ABCG2⁻ | 1,000,000 | 4/4 | 21 |
| | | | 100,000 | 5/5 | 22 |
| | | | 10,000 | 8/9 | 29 |
| | | | 1,000 | 5/9 | 30 |
| | | | 100 | 1/5 | 66 |
| CT3 | P3 | CD44^{hi} | 1,000 | 5/5 | 31# |
| | | | 100 | 5/5 | 34# |
| | | | 10 | 2/5 | 54 |
| | | CD44⁻ | 1,000 | 5/5 | 37 |
| | | | 100 | 2/5 | 36 |
| | | | 10 | 0/5 | |
| | | PI⁻ | 10,000 | 2/2 | 29 |
| | | | 1,000 | 5/5 | 38 |
| | | | 100 | 5/5 | 42 |
| | | | 10 | 1/4 | 59 |
| CT4 | P2 | CD44^{hi} | 10,000 | 2/2 | 141 |
| | | | 1,000 | 1/4 | 132 |
| | | | 100 | 0/4 | |
| | | CD44⁻ | 10,000 | 0/2 | |
| | | | 1,000 | 0/4 | |
| | | | 100 | 0/4 | |
| | | PI⁻ | 10,000 | 0/2 | |
| | | | 1,000 | 0/4 | |
| | | | 100 | 0/4 | |
| | | CD44^{hi} | 10,000 | 2/2 | 72 |
| | | CD26⁻ | | | |
| | | | 1,000 | 0/4 | |
| | | | 100 | 1/4 | 91 |
| CT5 | P2 | CD44^{hi} | 50,000 | 2/2¥ | 25 |
| | | | 10,000 | 4/4 | 35 |
| | | | 1,000 | 5/5 | 39 |
| | | CD44⁻ | 50,000 | 2/2 | 43 |
| | | | 10,000 | 1/4 | 44 |
| | | | 1,000 | 0/5 | |
| | | CD44^{hi} | 500 | 2/3 | 50 |
| | | ALDH⁺ | | | |
| | | | 100 | 5/5 | 65 |
| | | CD44^{hi} | 500 | 0/2 | |
| | | ALDH⁻ | | | |
| | | | 100 | 0/4 | |
| CT17 | P3 | CD44^{hi} | 1,000 | 0/4 | |
| | | | 100 | 0/4 | |
| | | CD44⁻ | 1,000 | 0/3 | |
| | | | 100 | 0/3 | |
| | | PI⁻ | 1,500 | 0/3 | |
| CT17 | P2 | CD44^{hi} | 1,000 | 0/4 | |
| | | | 100 | 0/4 | |
| | | CD44⁻ | 1,500 | 0/4 | |
| | | | 100 | 0/3 | |
| | | CD44^{hi} | 100 | 1/4 | 21 |
| | | CD133⁺ | | | |
| | | | 10 | 0/4 | |
| | | CD44^{hi} | 100 | 0/4 | |
| | | CD133⁻ | | | |
| | | | 10 | 0/4 | |
| | | CD133⁺ | 1,000 | 0/3 | |
| | | | 100 | 0/4 | |
| | | | 10 | 0/4 | |
| | | CD133⁻ | 1,000 | 0/2 | |
| | | | 100 | 0/4 | |
| | | | 10 | 0/4 | |

P(n), Passage number, where n = the number of passages.
PI⁻, cells that did not stain positive with propidium iodide (i.e. live cells), unsorted.
Tumor latency refers to the average time in days for a palpable tumor to be detected.
*Mean tumor latency for CT2 CD44^{hi} or ABCG2^{hi} cells was significantly shorter than matched CD44⁻ (p = 0.0001) or ABCG2⁻ cells (p = 0.0006), respectively; tumor latency for both groups was also significantly different when compared with parental unsorted PI⁻ cells (p = 0.0004).
#Mean tumor latency for CT3 CD44^{hi} tumors in 1,000 and 100 cell groups was significantly different than mean tumor latency for parental unsorted PI⁻ CT3 cells, p = 0.02 and p = 0.005, respectively.
¥Mean tumor latency for 50,000 CT5 CD44^{hi} cells was significantly different than mean tumor latency for 50,000 CD44⁻ cells, p = 0.05.

Using cells isolated from the CT2 primary tumor xenograft, all mice except one that were injected with 100,000 or more cells formed tumors, with no significant differences seen between CD44^{hi}, CD44⁻, ABCG2^{hi}, ABCG2⁻ and live unsorted subpopulations. However, when fewer than 100,000 cells from CT2 were implanted, the frequency of tumor formation was higher for CD44^{hi} and ABCG2^{hi} cells, compared to matched CD44⁻ and ABCG2⁻ cells. For example, 9/9 mice implanted with 10,000 CD44^{hi} cells had tumors at day 26, compared to only 1/9 and 2/9 mice implanted with CD44⁻ and unsorted parental cells, respectively. One thousand (1,000) CD44^{hi} cells formed tumors in 9/9 mice, compared to 3/9 and 4/9 mice with tumors from matched live unsorted and CD44⁻ cells, respectively (6 month follow-up). Finally, as few as 100 CD44^{hi} cells from CT2 formed tumors in 2/5 mice within 31 days, compared to no tumor formation from matched CD44⁻ and unsorted cells followed for up to 6 months.

ABCG2^{hi} cells from CT2 were also highly tumorigenic, with 8/9 mice forming tumors when implanted with 1,000 ABCG2^{hi} cells compared to 5/9 mice forming tumors when implanted with 1,000 ABCG2⁻ cells, at day 26. The difference in tumor forming ability between these two groups was more pronounced when 100 cells were implanted; 100 ABCG2^{hi} cells formed tumors in 5/9 mice, compared to 1/5 mice with tumors when implanted with matched live unsorted or ABCG2⁻ cells.

The enriched tumor forming ability of CD44^{hi} cells was reproduced with isolated cells from a second primary tumor xenograft, CT3. Implantation of 100 CD44^{hi} CT3 cells formed tumors in 5/5 mice, versus 2/5 mice forming tumors when injected with 100 CD44⁻ CT3 cells. 10 CD44^{hi} from CT3 formed tumors in 2/5 mice, whereas 0/5 mice formed tumors when implanted with 10 matched CD44⁻ cells.

CD44^{hi} and ABCG2^{hi} primary tumor xenograft cells also formed tumors with significantly shorter latency and significantly more aggressive growth. For example, when 10,000 cells were implanted, both CD44^{hi} and ABCG2^{hi} CT2 cells formed tumors with an average latency of 23 days. In contrast, the average tumor latency when 10,000 CD44⁻, ABCG2⁻, or live unsorted CT2 cells were implanted was significantly longer, i.e., 33, 29, and 28 days, respectively (p<0.004). Similarly, the average tumor latency of 100 CD44^{hi} cells from the CT3 primary tumor xenograft was shorter than CD44⁻ or live unsorted CT3 cells.

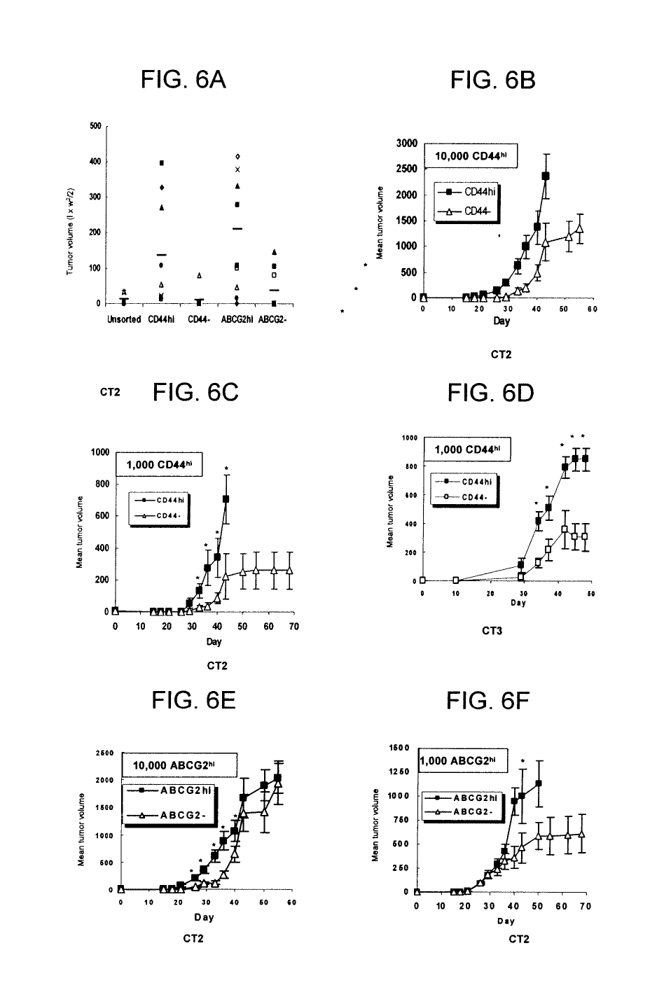

In addition to having an enhanced ability to form tumors at low cell numbers and with shorter latency, CD44^{hi} and ABCG2^{hi} cells from CT2 and CT3 also formed tumors that grew significantly more aggressively (FIGS. 6B-6D). Moreover, the final or maximum tumor volume resulting from implantation of CD44^{hi} cells was consistently larger than that of CD44⁻ cells. For example, 8/9 mice injected with 10,000 CD44^{hi} CT2 cells formed tumors achieving dimensions of greater than 1,900 mm³ by day 55, whereas only 3/9 mice injected with 10,000 CD44-cells formed tumors that reached 1900 mm³, even when followed for up to 6 months (FIG. 6B). Similarly, CD44^{hi} CT3 tumor cells formed tumors with mean volumes that were approximately 2-fold or more larger than tumors derived from CD44⁻ cells (FIG. 6D). Collectively, these observations indicate that CD44^{hi} and ABCG2^{hi} colon tumor cells are highly enriched for in vivo tumorigenicity. Although CD44⁻ and ABCG2⁻ cells also formed tumors, they show a limited proliferative potential compared to matched CD44^{hi} and ABCG2^{hi} counterparts.

In subsequent experiments, CD44^{hi} cells from tumor samples CT4 and CT5 were enriched for high tumorigenicity at low cell input numbers. CD44^{hi} cells isolated from CT4 and CT5 were tumorigenic at 1,000 and 10,000 cells, with a combined total of 12/15 mice forming tumors; in contrast, 1,000 and 10,000 CD44⁻ cells from both CT4 and CT5 were essentially non-tumorigenic, with tumor formation in only 1/15 mice. With CT17, no tumors formed from either live unsorted or CD44 sorted cells. This may be due to the fact that this primary xenograft grew very slowly even when implanted as whole tumor fragments, and the highest number of cells implanted per group (1,000 cells) in this experiment was not enough for tumor formation.

CD44^{hi} cells isolated from tumor sample CT5 were also enriched for expression of aldehyde dehydrogenase (ALDH). Aldehyde dehydrogenase (ALDH) has been previously described as a marker of neural stem cells (Corti et al., Stem Cells, 2006, 24(4):975-985). CD44^{hi} ALDH⁺ cells were tumorigenic at 500 and 100 cells, with a combined total of 7/8 mice forming tumors; in contrast, no tumors formed from CD44^{hi} ALDH⁻ cells.

Isolated CD44^{hi} colon tumor cells from four out of five primary patient samples tested were highly tumorigenic at low cell numbers in immune deficient mice. CD44^{hi} cells were about 10-fold to about 50-fold more tumorigenic at limiting cell numbers, as determined by comparing the number of CD44^{hi} versus CD44⁻ cells from the same patient sample needed to achieve the same frequency of tumor formation.

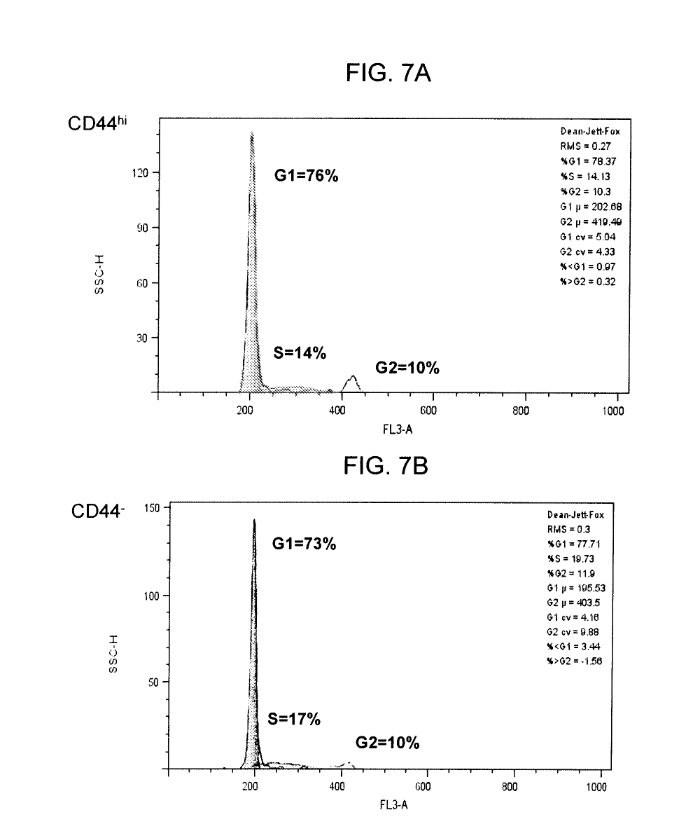

When mice were implanted with higher numbers of CT2, CT3, and CT5 cells, i.e. 10,000 cells or greater, most mice eventually formed tumors irrespective of CD44 status (CD44⁻ cells from CT4 were non-tumorigenic even at 10,000 cells). However, in these situations, CD44^{hi} cells consistently formed tumors with significantly shorter latency, more aggressive growth, and larger tumor volume than matched unsorted or CD44⁻ cells (FIGS. 6A and 6D). Although CD44^{hi} cells generated tumors with a highly proliferative growth rate, cell cycle analysis of colon tumor cells from CT2, CT4, and CT5 revealed no significant differences in the cell cycle status between sorted CD44^{hi} and CD44⁻ (FIGS. 7A-7B). This indicates that CD44^{hi} sorted cells were not preferentially cycling at the time of implant, but generated highly proliferative cells after in vivo injection.

### Example 5

### CD44^{hi} Colon Tumor Cells Regenerate Tumors Having Similar Histology and Gene Expression as the Parental Tumor

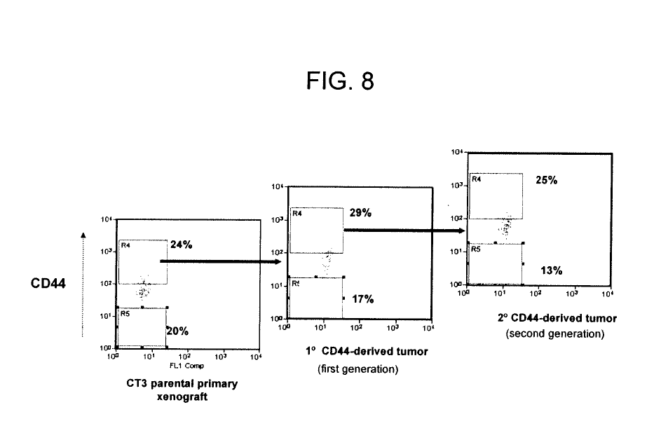

CD44^{hi} colon tumor cells have self-renewal capacity and regenerated the heterogeneous CD44^{hi} and CD44⁻ phenotype of the parent tumor. Tumors derived from isolated CD44^{hi} cells were dissociated and analyzed by flow cytometry. The secondary CD44-derived tumor expressed both CD44^{hi} and CD44⁻ cells with the same broad distribution of CD44 expression seen in the parental primary tumor. See FIG. 8, compare parental primary tumor with 1° CD44^{hi} derived tumor. The percentage of CD44^{hi} cells was consistent, staying within a range of approximately 25-33% for both parental tumors and those formed from serial transplantations. This finding was consistent in at least 6 separate experiments analyzing CD44 expression in tumors derived from CD44^{hi} CT2, CT3, CT4, and CT5 primary tumor xenograft cells (CT5 shown in FIGS. 5A-5F).

In serial transplantation experiments designed to test for self-renewal, 100 CD44^{hi} cells re-isolated from CD44^{hi} derived tumors (1° tumor) successfully formed secondary tumors in 4/5 mice, whereas no tumors formed in 5 mice implanted with 100 CD44 cells. These tumors had the same latency as the 1° or 1st generation tumors generated from 100 CD44^{hi} cells (approximately 34-37 days), and showed the same heterogeneous CD44 expression phenotype seen in the original parental xenograft and in the primary tumor. See FIG. 8 (1° and 2° CD44-derived tumors). CD44^{hi} cells successfully passaged through 4 rounds of serial implantation consistently formed tumors with no apparent loss of tumorigenicity. In 18/19 animals, tumors were observed at 4-6 weeks post-implantation in animals receiving 100 CD44^{hi} CT3 cells, while tumors formed in only 3/19 mice injected with 100 CD44⁻ cells. See Table 4.

Additionally, 500 and 100 CD44^{hi} ALDH⁺ cells re-isolated from CD44^{hi} ALDH⁺ derived tumors (1° tumor) successfully formed secondary tumors in 3/5 and 1/5 mice, respectively, whereas no secondary tumors formed in 10 mice implanted with 500 or 100 CD44^{hi} ALDH⁻. See Table 5.

In these serial transplantation experiments, 100 CD44^{hi} cells re-isolated from CD44^{hi} CT3 derived tumors successfully formed secondary tumors in 5/5 mice within 34 to 40 days (Table 4). In contrast, only 1/5 mice implanted with 100 CD44⁻ cells eventually formed a tumor at day 60 with a four month follow up. In subsequent serial transplantation experiments, 100 CD44^{hi} cells formed tertiary and quaternary tumors in 4/5 and 4/4 mice respectively, compared to 0/5 and 0/4 mice forming tumors with 100 CD44⁻ cells in these two experiments. In 5/5 mice also formed tertiary tumors when implanted with only 10 CD44^{hi} CT3 cells. This is consistent with earlier experiments where 2/5 mice formed tumors with 10 cells, and overall, a total of 7/10 mice implanted with 10 CD44^{hi} CT3 colon tumor cells successfully formed tumors. The ability of CD44^{hi} cells to be serially passaged was confirmed with patient sample CT5, in which CD44^{hi} cells were successfully transferred through three rounds of 1,000 cell transplants (Table 4). Thus, CD44^{hi} but not CD44⁻ colon tumor cells are enriched for the presence of cancer stem cells with the capacity for self-renewal.

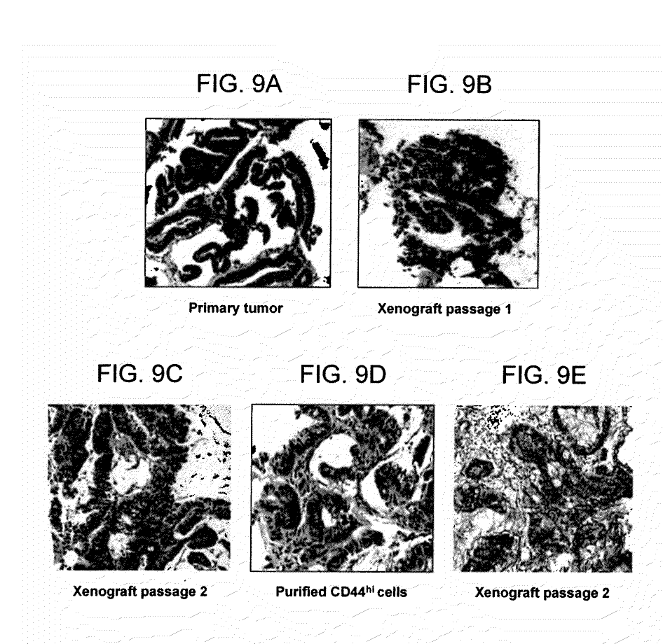

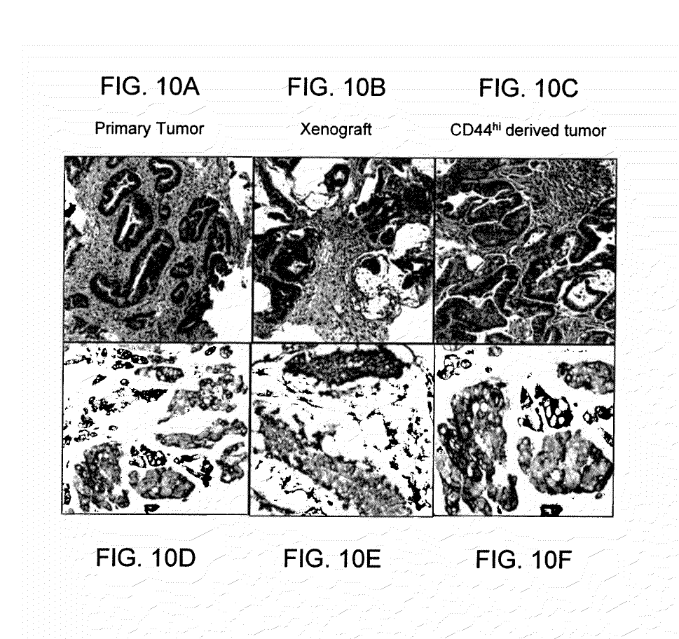

Hematoxylin and eosin stained sections of tumors from parental primary tumor xenografts used for in vivo tumorigenicity experiments were compared with tumors generated from sorted cells. Tumors formed from 10 and 100 CD44^{hi} CT2 and CT3 primary xenograft cells, respectively, and had a poorly differentiated histological appearance, similar to the original parental CT2 and CT3 primary tumors. Subcutaneous implantation of 1,000 and 10,000 isolated CD44^{hi} single cells from CT4 and CT5 primary xenograft tumors generated moderately differentiated primary tumors with similar histology to the moderately differentiated primary tumors and tumor xenografts from which they were derived (FIGS. 9A-9E and 10A-10C).

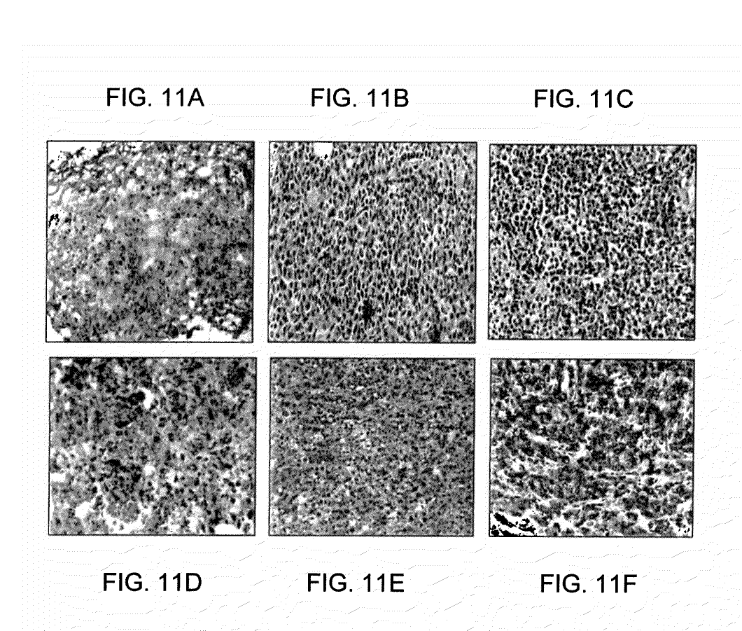

Similarly, tumors formed by low numbers of CD44^{hi} cells (10 to 1,000 cells) isolated from either poorly differentiated or moderately differentiated primary adenocarcinomas generated xenografts that recapitulated the same histologic features (i.e., gland formation, expression of CEA, and expression of cytokeratin 20) of the original CT3, CT4, and CT5 primary xenograft tumors (CT4, FIGS. 10A-10F; CT3, FIGS. 11A-11F). Thus, a glandular tumor mass was dissociated into single cells, CD44^{hi} cells were isolated and used to regenerate a tumor mass with glandular structures resembling those of the primary patient tumor. In addition, expression of CEA, cytokeratin 20, and AE1/AE3, markers commonly expressed on colon tumor cells, was also maintained throughout xenograft passage (FIGS. 10D-10E and FIGS. 11A-11B). The observation that very low numbers of CD44^{hi} colon tumor cells can be serially passaged, and form experimental xenograft tumors that resemble the original primary tumors, indicate that the CD44^{hi} subpopulation is preferentially enriched for cells that can initiate and sustain the growth of a primary tumor in vivo.

TABLE 4
Serial Transplantation of CD44^{hi} Colon Tumor Cells

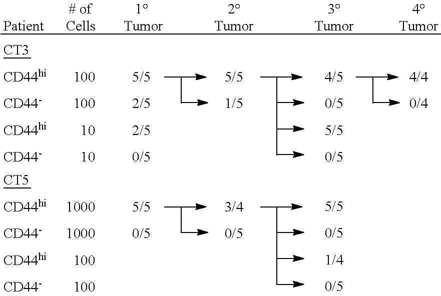

TABLE 5
CD44^{hi} ALDH⁺ Secondary Serial Transplant (patient CT5)

| Marker | # of Cells | Tumor Frequency | Tumor Latency |
|---|---|---|---|
| CD44^{hi} ALDH⁺ | 10 | 0/5 | 0 |
| | 100 | 1/5 | 48 |
| | 500 | 3/5 | 55 |
| CD44^{hi} ALDH⁻ | 10 | 0/5 | |
| | 100 | 0/5 | |
| | 500 | 0/5 | |
| CD44⁻ | 500 | 0/5 | |

### Example 6

### Additional Markers for Isolation and Enrichment of Cancer Stem Cells

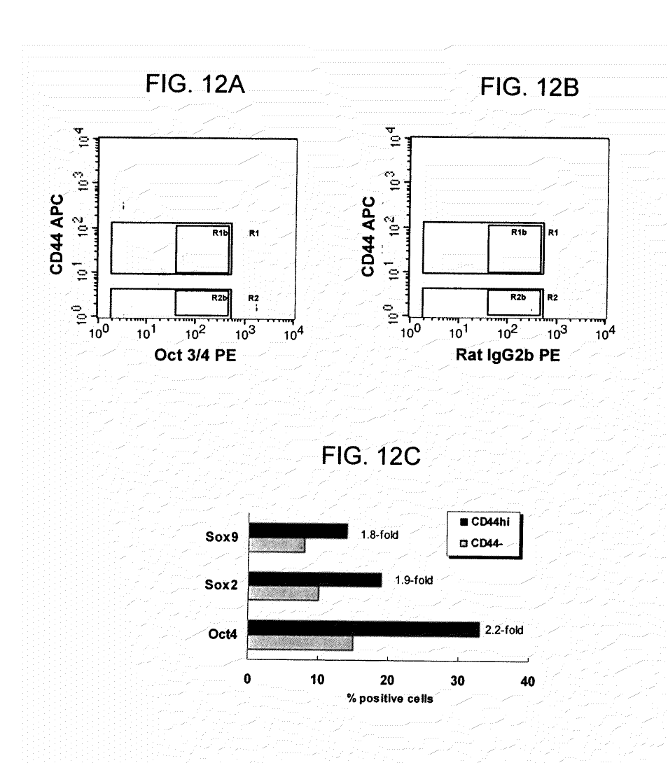

Primary colon tumor xenograft cells (CT3x) were depleted of mouse lineage cells by MOFLO® sort, stained to detect CD44 and adenomatous polyposis coli (APC) tumor suppressor, then fixed and permeabilized for intracellular staining with anti-Oct3/4-PE, anti-Sox-2-PE, anti-Sox-9, and matched isotype control antibodies. Anti-Sox-9 or isotype control goat IgG was detected using a secondary phycoerythrin (PE)-labeled anti-goat antibody. FACs analysis was performed essentially as in Example 1. FIGS. 12A-12B show FACs analysis of CD44 APC and isotype control (FIG. 12A) or Oct-3/4-PE labeled cells (FIG. 12B), with gating of CD44⁺ and CD44⁻ cells, and subgating of Oct-3/4⁺ cells. Co-expression of CD44 with each of Sox-2 and Sox-9 was similarly assayed. FIG. 12C shows the percentage of Oct-3/4, Sox-2, and Sox-9 cells that also express CD44, and the fold increase in the number of co-expressing cells as compared to the number of Oct-3/4, Sox-2, and Sox-9 positive cells that don't express CD44.

CD44^{hi} cells also coexpress VLA-2 (a receptor for ADAM9-S) and β-catenin, both of which are implicated in tumor invasion and liver metastasis of colon cancer. β-catenin is known to be essential for maintaining the multipotent stem-like nature of normal colon stem cells, and is also known to be activated in many cancers including colon cancer. CD44^{hi} cells obtained from two primary colon tumor xenografts (from patients CT2 and CT5) are enriched for nuclear β-catenin. CD44^{hi} cells were sorted, fixed, and stained with an anti-β-catenin antibody and counterstained with the nuclear specific DAPI stain. CD44^{hi} cells showed a high coincidence of β-catenin and DAPI staining, whereas many CD44⁻ cells lacked β-catenin expression. Co-localization with nuclear DAPI stain demonstrated that the vast majority of β-catenin staining was localized to the nucleus, although cytoplasmic staining was also seen. Some CD44⁻ cells also stained positive for β-catenin, although nuclear staining of CD44⁻ cells was less prominent. Scoring of 10 random fields from β-catenin-labeled cells revealed that nuclear β-catenin was detected in 124/160 (78%) and 159/236 (67%) CD44^{hi} cells from CT2 and CT5, respectively, as compared to only 33/184 (18%) and 48/404 (12%) of CD44⁻ cells from matched CT2 and CT5 controls, respectively.

The tumorigenicity of CD44^{hi} cells was enriched by further depleting the CD44^{hi} population of cells expressing CD26, a differentiation marker expressed on colon columnar cells. Tumors derived from CD44^{hi} CD26⁻ cells do express CD26, further supporting that the CD44^{hi} CD26⁻ cancer stem cells are capable of differentiation.

The expression of CD133 was studied in colon tumor cells using FACS cell sorting as described in Example 1. Patient samples were analyzed either before or after xenograft passage in immune deficient mice. CD133⁺ cells were identified in primary colon tumor samples CT3, CT4, CT7-9, CT12, and CT21. Some of these CD133⁺ colon tumor cells were also CD44^{hi}; CT3, CT4, CT7, and CT21. See Table 6.

TABLE 6
Percentage of CD133-Expressing Colon Tumor Cells

| Patient | CD133⁺ | CD44^{hi} CD133⁺ |
|---|---|---|
| CT3 | 1-2 | 0.2-0.8 |
| CT4 | 4 | 2 |
| CT7 | 2-4 | 1.3 |
| CT8 | 3-5 | ND |
| CT9 | 0.5-2 | ND |
| CT12 | 30 | ND |
| CT21 | 0.3-1 | 0.2 |

ND = not determined

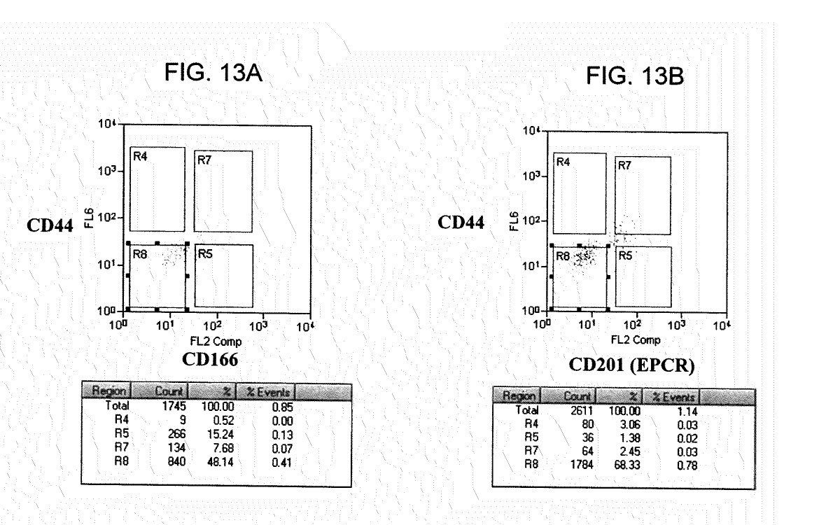

CD166 and CD201 (endothelial protein C receptor, EPCR) are also expressed on primary colon tumor xenograft cells and are co-expressed with the CD44^{hi} population. Expression of CD166 and CD201 on colon tumor cells was analyzed by flow cytometry as described herein. See FIGS. 13A-13B and Table 7 below.

TABLE 7
Expression of CD166 and CD201 on Colon Tumor Cells

| Patient | CD166⁺ | CD44^{hi} CD166⁺ | CD201⁺ | CD44^{hi} CD201⁺ |
|---|---|---|---|---|
| CT3 | 11.5% | 9.0% | 11.0% | 7.7% |
| CT4 | 23% | 7.7% | 7.0% | 2.5% |

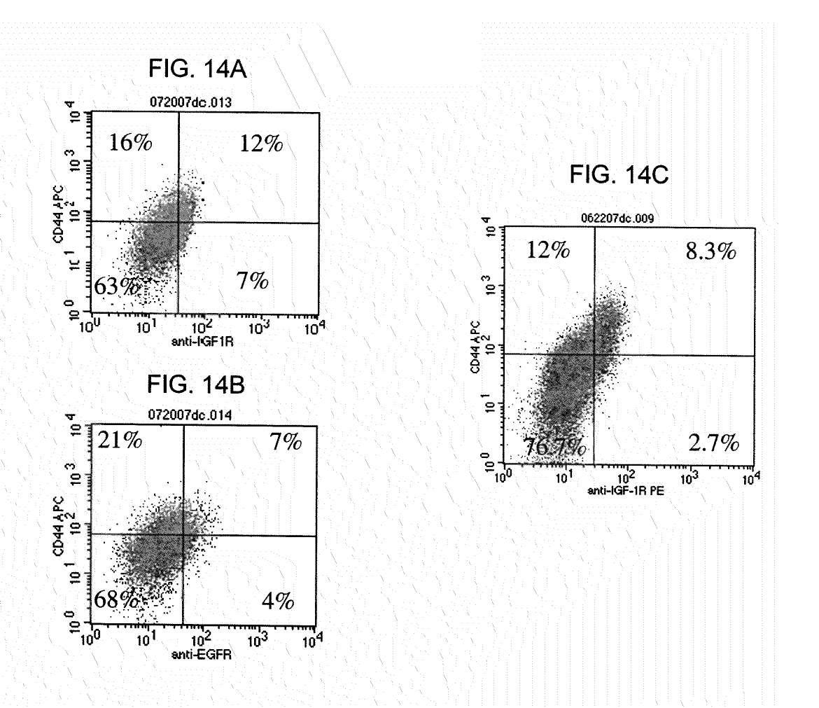

CD44^{hi} colon tumor cells also co-express IGF-1R and EGF-R, as determined by flow cytometry analyses described herein. See FIGS. 14A-14C. In particular, a majority of colon cancer cells expressing IGF1R or EGFR also express CD44^{hi}. Expression of these tumor growth factor receptors on CD44^{hi} colon tumor cells is consistent with the highly proliferative potential of the CD44^{hi} tumor cells.

Additional potential markers for cancer stem cells are selected based upon expression in CD44^{hi} cells as determined by differential expression analysis. For isolation or enrichment of a cancer stem cell population as described herein, potential markers identified by differential expression analyses are additionally characterized by expression of the corresponding proteins at the cell surface such that they are amenable to cell sorting techniques. Useful markers include proteins encoded by genes that show measurable expression that is increased (i.e., upregulated) or decreased (i.e., downregulated) in CD44^{hi} cells as compared to CD44⁻ cells. Thus, for selection of cancer stem cells, both detectable expression (i.e., positive expression) and/or levels of expression in CD44^{hi} versus CD44⁻ cells may be used as selection criteria.

For differential expression analysis, cells were obtained from CT21 primary tumor cells and sorted according to CD44^{hi}/CD44⁻ expression as described in Example 1. Cells were sorted into multiple replicates, such that the CD44^{hi} population was obtained from 3 replicate cell sorting analyses, and the CD44⁻ population was obtained from 7 replicate cell sorting analyses. A human expression analysis array (Human Gene Plus 2 Array was purchased from Affymetrix (Santa Clara, Calif.) and hybrized to probes prepared from the CD44^{hi} and CD44⁻ populations. Probe intensities were normalized using GCRMA method. Gene expression values were estimated using linear models and pre-defined groups. Genes differentially expressed in the CD44^{hi} and CD44⁻ populations were identified using multivariate analysis and Bayesian log-odds posterior probabilities (B lods) as known in the art. When compared to baseline values obtained from the CD44⁻ population, genes were identified as differentially expressed if B lods 1.5 and βFC|≧2 and present (i.e., reliably detected in at least half of the replicates for at least one of the CD44^{hi} group or CD44⁻ group). A B lods score of 1.5 indicates 82% probability that the gene is differentially expressed. Differentially expressed genes are listed in Table 8. Among the differentially expressed genes were SPARC (Osteonectin), COL1A1 (Collagen, type I, alpha I), ID3 (Inhibitor of DNA binding 4), ID4 (Inhibitor of DNA binding 4), and CDKN1a (8 IDs for 5 genes), whose expression is also described in Shipitsin et al., Cancer Cell, 2007, 11:259-273.

TABLE 8
Genes That Are Differentially Expressed in CD44^{hi} and CD44⁻ Populations

| | Fold change score |
|---|---|
| | >100 = +++ |
| | 10-99 = ++ |
| | 2-9 = + |
| Genes Upregulated in CD44^{hi} Colon Tumor Cells | |
| Compared to CD44⁻ Cells | |
| interferon, alpha-inducible protein 6 /// immunoglobulin heavy locus /// | +++ |
| immunoglobulin heavy constant gamma 1 (G1m marker) /// | |
| immunoglobulin heavy constant gamma 2 (G2m marker) /// | |
| immunoglobulin heavy constant gamma 3 (G3m marker) /// | |
| immunoglobulin heavy constant mu /// immunoglobulin heavy variable 4-31 | |
| matrix metallopeptidase 1 (interstitial collagenase) | +++ |
| immunoglobulin kappa constant /// immunoglobulin kappa variable 1-5 /// | +++ |
| immunoglobulin kappa variable 2-24 | |
| Major histocompatibility complex, class I, C | +++ |
| collagen, type III, alpha 1 (Ehlers-Danlos syndrome type IV, autosomal | +++ |
| dominant) | |
| decorin | ++ |
| matrix metallopeptidase 3 (stromelysin 1, progelatinase) | ++ |
| immunoglobulin heavy constant delta | ++ |
| matrix-remodelling associated 5 | ++ |
| asporin | ++ |
| matrix metallopeptidase 9 (gelatinase B, 92 kDa gelatinase, 92 kDa type IV | ++ |
| collagenase) | |
| immunoglobulin kappa constant /// immunoglobulin kappa variable 1-5 | ++ |
| ADAM-like, decysin 1 | ++ |
| collagen, type XII, alpha 1 | ++ |
| periostin, osteoblast specific factor | ++ |
| cathepsin K | ++ |
| collagen, type V, alpha 1 | ++ |
| versican | ++ |
| sulfatase 1 | ++ |
| lumican | ++ |
| major histocompatibility complex, class II, DP beta 1 | ++ |
| RAB31, member RAS oncogene family | ++ |
| fibulin 1 | ++ |
| collagen, type I, alpha 2 | ++ |
| CDNA FLJ11041 fis, clone PLACE1004405 | ++ |
| anthrax toxin receptor 1 | ++ |
| chemokine (C—X—C motif) ligand 14 | ++ |
| platelet-derived growth factor receptor, alpha polypeptide | ++ |
| major histocompatibility complex, class II, DP alpha 1 | ++ |
| follistatin-like 1 | ++ |
| nicotinamide N-methyltransferase | ++ |
| solute carrier family 2 (facilitated glucose transporter), member 3 | ++ |
| matrix metallopeptidase 12 (macrophage elastase) | ++ |
| caldesmon 1 | ++ |
| collagen, type VI, alpha 3 | ++ |
| insulin-like growth factor binding protein 5 | ++ |
| secreted phosphoprotein 1 (osteopontin, bone sialoprotein I, early T- | ++ |
| lymphocyte activation 1) | |
| Fc fragment of IgE, high affinity I, receptor for; gamma polypeptide | ++ |
| annexin A1 | ++ |
| thrombospondin 2 | ++ |
| cadherin 11, type 2, OB-cadherin (osteoblast) | ++ |
| gap junction protein, alpha 1, 43 kDa | ++ |
| complement component 1, s subcomponent | ++ |
| major histocompatibility complex, class II, DQ alpha 1 | ++ |
| Transcribed locus | ++ |
| killer cell lectin-like receptor subfamily C, member 1 /// killer cell lectin-like | ++ |
| receptor subfamily C, member 2 | |
| podoplanin | ++ |
| CD53 molecule | ++ |
| similar to Complement C3 precursor | ++ |
| protocadherin 18 | ++ |
| pleckstrin homology domain containing, family C (with FERM domain) | + |
| member 1 | |
| secreted protein, acidic, cysteine-rich (osteonectin) | ++ |
| collagen, type I, alpha 1 | ++ |
| collagen, type V, alpha 2 | ++ |
| EGF-like repeats and discoidin I-like domains 3 | ++ |
| membrane-spanning 4-domains, subfamily A, member 6A | ++ |
| lysosomal associated multispanning membrane protein 5 | ++ |
| major histocompatibility complex, class II, DR alpha | + |
| sal-like 1 (Drosophila) | + |
| heat shock 70 kDa protein 1A | + |
| distal-less homeobox 2 | + |
| fibronectin type III domain containing 1 | + |
| collagen, type I, alpha 1 | + |
| tumor necrosis factor, alpha-induced protein 6 | + |
| death-associated protein kinase 1 | + |
| matrix metallopeptidase 2 (gelatinase A, 72 kDa gelatinase, 72 kDa type IV | + |
| collagenase) | |
| lectin, galactoside-binding, soluble, 1 (galectin 1) | + |
| major histocompatibility complex, class II, DQ alpha 1 /// major | + |
| histocompatibility complex, class II, DQ alpha 2 /// similar to HLA class II | |
| histocompatibility antigen, DQ(1) alpha chain precursor (DC-4 alpha | |
| chain) | |
| DEP domain containing 1 | + |
| SHC SH2-domain binding protein 1 | + |
| vascular cell adhesion molecule 1 | + |
| AE binding protein 1 | + |
| aldehyde dehydrogenase 1 family, member L2 | + |
| family with sequence similarity 26, member F | + |
| biglycan | + |
| interleukin 1 receptor, type II | + |
| coagulation factor II (thrombin) receptor-like 2 | + |
| tenascin C (hexabrachion) | + |
| kallikrein-related peptidase 7 | + |
| hypothetical protein LOC340340 | + |
| interleukin 33 | + |
| Zwilch, kinetochore associated, homolog (Drosophila) | + |
| MHC class I polypeptide-related sequence B | + |
| potassium channel tetramerisation domain containing 12 | + |
| peripheral myelin protein 22 | + |
| FYN oncogene related to SRC, FGR, YES | + |
| AXL receptor tyrosine kinase | + |
| fibrinogen-like 2 | + |
| cysteine-rich, angiogenic inducer, 61 | + |
| transmembrane protein 45A | + |
| msh homeobox 2 | + |
| cysteine-rich protein 1 (intestinal) | + |
| cystatin A (stefin A) | + |
| malic enzyme 1, NADP(+)-dependent, cytosolic | + |
| tubulin, beta 6 | + |
| Transcribed locus | + |
| BUB1 budding uninhibited by benzimidazoles 1 homolog (yeast) | + |
| serum amyloid A1 /// serum amyloid A2 | + |
| NIMA (never in mitosis gene a)-related kinase 2 | + |
| Transcribed locus | + |
| Rho GTPase activating protein 11A | + |
| Rho GDP dissociation inhibitor (GDI) beta | + |
| epithelial cell transforming sequence 2 oncogene | + |
| Transcribed locus | + |
| testis expressed 15 | + |
| ChaC, cation transport regulator homolog 2 (E. coli) | + |
| minichromosome maintenance complex component 10 | + |
| TIMP metallopeptidase inhibitor 2 | + |
| KIAA1524 | + |
| phosphoserine aminotransferase 1 | + |
| Similar to RIKEN cDNA 2310016C16 | + |
| macrophage expressed gene 1 | + |
| Fanconi anemia, complementation group B | + |
| BRCA1 interacting protein C-terminal helicase 1 | + |
| family with sequence similarity 64, member A | + |
| karyopherin alpha 3 (importin alpha 4) | + |
| apolipoprotein D | + |
| tripartite motif-containing 31 | + |
| dynein, axonemal, heavy chain 10 | + |
| runt-related transcription factor 2 | + |
| urothelial cancer associated 1 | + |
| centromere protein I | + |
| kinesin family member 14 | + |
| major histocompatibility complex, class II, DR beta 4 | + |
| Cell division cycle 2, G1 to S and G2 to M | + |
| Atonal homolog 8 (Drosophila) | + |
| CDNA clone IMAGE: 4822878 | + |
| chromosome 18 open reading frame 24 | + |
| Full-length cDNA clone CS0DI067YM20 of Placenta Cot 25-normalized of | + |
| Homo sapiens (human) | |
| DEP domain containing 1 /// similar to DEP domain containing 1 | + |
| collagen triple helix repeat containing 1 | + |
| Transcribed locus | + |
| complement factor I | + |
| chromosome 13 open reading frame 3 | + |
| aldo-keto reductase family 1, member C4 (chlordecone reductase; 3- | + |
| alpha hydroxysteroid dehydrogenase, type I; dihydrodiol dehydrogenase | |
| 4) | |
| v-myb myeloblastosis viral oncogene homolog (avian)-like 2 | + |
| anillin, actin binding protein | + |
| serum amyloid A1 | + |
| solute carrier family 16, member 14 (monocarboxylic acid transporter 14) | + |
| coiled-coil domain containing 3 | + |
| solute carrier family 7 (cationic amino acid transporter, y+ system), | + |
| member 2 | |
| centromere protein N | + |
| cyclin A2 | + |
| establishment of cohesion 1 homolog 2 (S. cerevisiae) | + |
| lysozyme (renal amyloidosis) | + |
| Arsenic transactivated protein 1 | + |
| family with sequence similarity 72, member A /// similar to family with | + |
| sequence similarity 72, member A | |
| primase, polypeptide 2A, 58 kDa | + |
| pro-melanin-concentrating hormone | + |
| DENN/MADD domain containing 1A | + |
| WD repeat domain 67 | + |
| centromere protein E, 312 kDa | + |
| kinesin family member 15 | + |
| baculoviral IAP repeat-containing 5 (survivin) | + |
| non-SMC condensin I complex, subunit H | + |
| polo-like kinase 1 (Drosophila) | + |
| carcinoembryonic antigen-related cell adhesion molecule 8 | + |
| pro-melanin-concentrating hormone-like 1 | + |
| shugoshin-like 1 (S. pombe) | + |
| tumor necrosis factor receptor superfamily, member 19 | + |
| DNA2 DNA replication helicase 2-like (yeast) | + |
| Transcribed locus | + |
| ribonucleotide reductase M1 polypeptide | + |
| BMP and activin membrane-bound inhibitor homolog (Xenopus laevis) | + |
| msh homeobox 1 | + |
| allograft inflammatory factor 1 | + |
| RAD51 associated protein 1 | + |
| hypothetical protein FLJ25416 | + |
| solute carrier family 7 (cationic amino acid transporter, y+ system), | + |
| member 7 | |
| zinc finger, RAN-binding domain containing 3 | + |
| interleukin 6 receptor | + |
| hypothetical protein LOC221362 /// similar to heterogeneous nuclear | + |
| ribonucleoprotein A/B | |
| carboxypeptidase, vitellogenic-like | + |
| maternal embryonic leucine zipper kinase | + |
| PDZ binding kinase | + |
| centromere protein K | + |
| glutathione peroxidase 3 (plasma) | + |
| Transcribed locus | + |
| apolipoprotein B mRNA editing enzyme, catalytic polypeptide-like 3B | + |
| Opa interacting protein 5 | + |
| SPC25, NDC80 kinetochore complex component, homolog (S. cerevisiae) | + |
| chromosome 12 open reading frame 48 | + |
| cell division cycle 25 homolog A (S. pombe) | + |
| cyclin E2 | + |
| aryl hydrocarbon receptor nuclear translocator-like 2 | + |
| chromosome 4 open reading frame 18 | + |
| nidogen 1 | + |
| Fanconi anemia, complementation group D2 | + |
| chloride channel 5 (nephrolithiasis 2, X-linked, Dent disease) | + |
| heat shock 70 kDa protein 4-like | + |
| TTK protein kinase | + |
| FLJ20105 protein | + |
| hyaluronan-mediated motility receptor (RHAMM) | + |
| Full length insert cDNA clone ZD90B10 | + |
| citron (rho-interacting, serine/threonine kinase 21) | + |
| replication factor C (activator 1) 3, 38 kDa | + |
| DEP domain containing 1B | + |
| stomatin | + |
| GLI pathogenesis-related 1 (glioma) | + |
| hydroxysteroid (17-beta) dehydrogenase 6 homolog (mouse) | + |
| WD repeat and HMG-box DNA binding protein 1 | + |
| biliverdin reductase A | + |
| G protein-coupled receptor 115 | + |
| structural maintenance of chromosomes 4 | + |
| major histocompatibility complex, class II, DM beta | + |
| kallikrein-related peptidase 10 | + |
| arachidonate 5-lipoxygenase-activating protein | + |
| crystallin, alpha B | + |
| cyclin-dependent kinase inhibitor 3 (CDK2-associated dual specificity | + |
| phosphatase) | |
| discs, large homolog 7 (Drosophila) | + |
| baculoviral IAP repeat-containing 3 | + |
| v-myb myeloblastosis viral oncogene homolog (avian)-like 1 | + |
| M-phase phosphoprotein 1 | + |
| tubulin, beta 2A /// tubulin, beta 2B | + |
| cyclin B1 | + |
| Fc fragment of IgG, low affinity IIa, receptor (CD32) | + |
| kinesin family member 4A | + |
| E2F transcription factor 8 | + |
| mannosidase, endo-alpha | + |
| acidic (leucine-rich) nuclear phosphoprotein 32 family, member E | + |
| FK506 binding protein 5 | + |
| chemokine (C—X—C motif) ligand 1 (melanoma growth stimulating activity, | + |
| alpha) | |
| diaphanous homolog 3 (Drosophila) | + |
| NUF2, NDC80 kinetochore complex component, homolog (S. cerevisiae) | + |
| structural maintenance of chromosomes 2 | + |
| neuritin 1 | + |
| galanin | + |
| claudin 2 | + |
| zinc finger protein 367 | + |
| Lin-7 homolog A (C. elegans) | + |
| furry homolog (Drosophila) | + |
| cytoskeleton associated protein 2 | + |
| NDC80 homolog, kinetochore complex component (S. cerevisiae) | + |
| chromosome 15 open reading frame 23 | + |
| Inhibitor of DNA binding 4, dominant negative helix-loop-helix protein | + |
| RAD54 homolog B (S. cerevisiae) | + |
| minichromosome maintenance complex component 8 | + |
| kinesin family member 23 | + |
| SLIT-ROBO Rho GTPase activating protein 2 | + |
| similar to 4931415M17 protein | + |
| cell division cycle associated 3 | + |
| cell division cycle associated 8 | + |
| serpin peptidase inhibitor, clade A (alpha-1 antiproteinase, antitrypsin), | + |
| member 3 | |
| G-2 and S-phase expressed 1 | + |
| non-SMC condensin I complex, subunit G | + |
| Protease, serine, 23 | + |
| kelch-like 5 (Drosophila) | + |
| GINS complex subunit 1 (Psf1 homolog) | + |
| kinesin family member 20A | + |
| family with sequence similarity 83, member D | + |
| forkhead box M1 | + |
| tensin 4 | + |
| nei endonuclease VIII-like 3 (E. coli) | + |
| CHK1 checkpoint homolog (S. pombe) | + |
| filamin A, alpha (actin binding protein 280) | + |
| pleckstrin homology domain containing, family K member 1 | + |
| thymidine kinase 1, soluble | + |
| ATG3 autophagy related 3 homolog (S. cerevisiae) | + |
| cell division cycle 7 homolog (S. cerevisiae) | + |
| CDNA FLJ33585 fis, clone BRAMY2012163 | + |
| Transcribed locus | + |
| inhibitor of DNA binding 3, dominant negative helix-loop-helix protein | + |
| protein regulator of cytokinesis 1 | + |
| chromosome 18 open reading frame 54 | + |
| CDNA FLJ23692 fis, clone HEP10227 | + |
| paraneoplastic antigen MA2 | + |
| glutathione S-transferase, C-terminal domain containing | + |
| retinol binding protein 2, cellular | + |
| centromere protein A | + |
| family with sequence similarity 111, member B | + |
| PHD finger protein 20-like 1 | + |
| Transcribed locus | + |
| chromosome 5 open reading frame 34 | + |
| KIAA1913 | + |
| RAD18 homolog (S. cerevisiae) | + |
| aurora kinase B | + |
| caveolin 2 | + |
| Solute carrier family 39 (zinc transporter), member 8 | + |
| kinesin family member 11 | + |
| chromosome 1 open reading frame 135 | + |
| centrosomal protein 55 kDa | + |
| SPC24, NDC80 kinetochore complex component, homolog (S. cerevisiae) | + |
| annexin A2 pseudogene 1 | + |
| Full-length cDNA clone CS0DI067YM20 of Placenta Cot 25-normalized of | + |
| Homo sapiens (human) | |
| hypothetical protein LOC728192 /// hypothetical protein LOC731880 | + |
| CDNA clone IMAGE: 6043059 | + |
| asp (abnormal spindle) homolog, microcephaly associated (Drosophila) | + |
| Full-length cDNA clone CS0DI067YM20 of Placenta Cot 25-normalized of | + |
| Homo sapiens (human) | |
| stress 70 protein chaperone, microsome-associated, 60 kDa | + |
| family with sequence similarity 54, member A | + |
| polymerase (DNA directed), theta | + |
| chromosome 6 open reading frame 173 | + |
| phosphoribosylglycinamide formyltransferase, phosphoribosylglycinamide | + |
| synthetase, phosphoribosylaminoimidazole synthetase | |
| RCD1 required for cell differentiation1 homolog (S. pombe) | + |
| ankyrin repeat and SOCS box-containing 4 | + |
| tubulin tyrosine ligase | + |
| transcription factor 19 (SC1) | + |
| SAM domain and HD domain 1 | + |
| hypothetical protein LOC146909 | + |
| polymerase (DNA directed), epsilon 2 (p59 subunit) | + |
| defective in sister chromatid cohesion homolog 1 (S. cerevisiae) | + |
| eukaryotic translation initiation factor 2 alpha kinase 4 | + |
| kallikrein-related peptidase 6 | + |
| protein tyrosine phosphatase, non-receptor type 2 | + |
| ring finger and CCCH-type zinc finger domains 1 | + |
| ATPase family, AAA domain containing 2 | + |
| thyroglobulin | + |
| adaptor-related protein complex 1, sigma 3 subunit | + |
| cancer susceptibility candidate 5 | + |
| dihydrofolate reductase | + |
| polo-like kinase 4 (Drosophila) | + |
| regulator of chromosome condensation 1 | + |
| KIAA1429 | + |
| shugoshin-like 2 (S. pombe) | + |
| ubiquitin-conjugating enzyme E2T (putative) | + |
| Bloom syndrome | + |
| progestin and adipoQ receptor family member VIII | + |
| hypothetical protein FLJ10781 | + |
| flap structure-specific endonuclease 1 | + |
| Transcribed locus | + |
| thyroid hormone receptor interactor 13 | + |
| BUB1 budding uninhibited by benzimidazoles 1 homolog beta (yeast) | + |
| IQ motif containing GTPase activating protein 3 | + |
| asparagine-linked glycosylation 10 homolog (yeast, alpha-1,2- | + |
| glucosyltransferase) | |
| La ribonucleoprotein domain family, member 4 | + |
| lamin B2 | + |
| T-box 3 (ulnar mammary syndrome) | + |
| prostate collagen triple helix | + |
| enolase 1, (alpha) | + |
| selenocysteine lyase | + |
| denticleless homolog (Drosophila) | + |
| ASF1 anti-silencing function 1 homolog B (S. cerevisiae) | + |
| helicase, lymphoid-specific | + |
| Transcribed locus | + |
| cryptochrome 1 (photolyase-like) | + |
| ribonucleotide reductase M2 polypeptide | + |
| cyclin B2 | + |
| origin recognition complex, subunit 5-like (yeast) | + |
| small optic lobes homolog (Drosophila) | + |
| ubiquitin specific peptidase 14 (tRNA-guanine transglycosylase) | + |
| ras homolog gene family, member Q | + |
| G protein-coupled receptor 107 | + |
| origin recognition complex, subunit 6 like (yeast) | + |
| importin 11 | + |
| myeloid/lymphoid or mixed-lineage leukemia (trithorax homolog, | + |
| Drosophila); translocated to, 11 | |
| WD repeat domain 76 | + |
| homeobox A10 | + |
| trophinin associated protein (tastin) | + |
| CDC45 cell division cycle 45-like (S. cerevisiae) | + |
| ATPase family, AAA domain containing 2 | + |
| DnaJ (Hsp40) homolog, subfamily A, member 4 | + |
| carbonic anhydrase XIII | + |
| chromosome 1 open reading frame 112 | + |
| PR domain containing 1, with ZNF domain | + |
| Mesenchymal stem cell protein DSC96 | + |
| cell division cycle 27 homolog (S. cerevisiae) | + |
| cleavage and polyadenylation specific factor 2, 100 kDa | + |
| cyclin-dependent kinase inhibitor 1A (p21, Cip1) | + |
| Growth arrest-specific 2 like 3 | + |
| catenin (cadherin-associated protein), alpha-like 1 | + |
| epiregulin | + |
| Homo sapiens, clone IMAGE: 3866695, mRNA | + |
| Full-length cDNA clone CS0DF025YM09 of Fetal brain of Homo sapiens | + |
| (human) | |
| cathepsin C | + |
| three prime histone mRNA exonuclease 1 | + |
| chromosome 1 open reading frame 43 | + |
| SMAD family member 7 | + |
| karyopherin alpha 2 (RAG cohort 1, importin alpha 1) | + |
| suppressor of variegation 3-9 homolog 2 (Drosophila) | + |
| aurora kinase A | + |
| non-SMC condensin II complex, subunit G2 | + |
| mutL homolog 1, colon cancer, nonpolyposis type 2 (E. coli) | + |
| topoisomerase (DNA) II alpha 170 kDa | + |
| chromosome 12 open reading frame 5 | + |
| NudE nuclear distribution gene E homolog 1 (A. nidulans) | + |
| centromere protein M | + |
| TPX2, microtubule-associated, homolog (Xenopus laevis) | + |
| nucleolar and spindle associated protein 1 | + |
| heparan sulfate 2-O-sulfotransferase 1 | + |
| ribonuclease, RNase A family, 1 (pancreatic) | + |
| GINS complex subunit 3 (Psf3 homolog) | + |
| transmembrane protein 180 | + |
| Splicing factor, arginine/serine-rich 2, interacting protein | + |
| Homo sapiens, clone IMAGE: 3897156, mRNA | + |
| deoxythymidylate kinase (thymidylate kinase) /// similar to | + |
| deoxythymidylate kinase (thymidylate kinase) | |
| CDNA FLJ34225 fis, clone FCBBF3023372 | + |
| dual specificity phosphatase 9 | + |
| progestin and adipoQ receptor family member III | + |
| CDNA clone IMAGE: 4797099 | + |
| isoprenylcysteine carboxyl methyltransferase | + |
| testis specific, 14 | + |
| MAD2 mitotic arrest deficient-like 1 (yeast) | + |
| family with sequence similarity 29, member A | + |
| KIAA0101 | + |
| mago-nashi homolog 2 | + |
| polymerase (RNA) III (DNA directed) polypeptide G (32 kD) | + |
| non-SMC condensin II complex, subunit D3 | + |
| homer homolog 1 (Drosophila) | + |
| phosphoglucomutase 2 | + |
| hypothetical protein FLJ40869 | + |
| chemokine (C—X—C motif) ligand 3 | + |
| Gens Downregulated in CD44^{hi} Colon Tumor Cells | |
| Compared to CD44⁻ Cells | |
| PTK2B protein tyrosine kinase 2 beta | + |
| insulin receptor substrate 1 | + |
| arylsulfatase D | + |
| uridine-cytidine kinase 1-like 1 | + |
| FLJ38717 protein | + |
| receptor-interacting serine-threonine kinase 3 | + |
| histone cluster 1, H2bd | + |
| insulin receptor substrate 2 | + |
| galactose-1-phosphate uridylyltransferase | + |
| calcium binding and coiled-coil domain 1 | + |
| calcium channel, voltage-dependent, beta 3 subunit | + |
| tight junction protein 3 (zona occludens 3) | + |
| endo-beta-N-acetylglucosaminidase | + |
| chromosome X open reading frame 10 | + |
| chromosome 10 open reading frame 99 | + |
| nuclear receptor subfamily 1, group H, member 3 | + |
| v-erb-b2 erythroblastic leukemia viral oncogene homolog 2, | + |
| neuro/glioblastoma derived oncogene homolog (avian) | |
| integrin, beta 4 | + |
| ceramide kinase | + |
| tetraspanin 5 | + |
| CDNA FLJ42259 fis, clone TKIDN2011289 | + |
| zinc finger and BTB domain containing 20 | + |
| transmembrane channel-like 4 | + |
| Transcribed locus | + |
| PDZK1 interacting protein 1 | + |
| N-acylsphingosine amidohydrolase (acid ceramidase)-like | + |
| SATB homeobox 1 | + |
| hypothetical protein MGC32805 | + |
| Similar to mitochondrial ribosomal protein L45 | + |
| hypothetical LOC440918 | + |
| nuclear receptor coactivator 1 | + |
| carboxyl ester lipase pseudogene | + |
| chromosome 10 open reading frame 81 | + |
| dedicator of cytokinesis 6 | + |
| chromosome 8 open reading frame 70 | + |
| epidermal growth factor receptor (erythroblastic leukemia viral (v-erb-b) | + |
| oncogene homolog, avian) | |
| endoplasmic reticulum to nucleus signalling 2 | + |
| KIAA1166 | + |
| loss of heterozygosity, 11, chromosomal region 2, gene A | + |
| hypothetical protein FLJ20209 | + |
| CDNA clone IMAGE: 5263455 | + |
| tRNA splicing endonuclease 2 homolog (S. cerevisiae) | + |
| Full length insert cDNA clone ZD55G10 | + |
| retinoblastoma binding protein 6 | + |
| hydroxypyruvate isomerase homolog (E. coli) | + |
| metastasis suppressor 1 | + |
| GEM interacting protein | + |
| F-box and leucine-rich repeat protein 6 | + |
| lectin, galactoside-binding, soluble, 9 (galectin 9) | + |
| Src homology 2 domain containing transforming protein D | + |
| WD repeat domain 19 | + |
| zinc finger protein 503 | + |
| hypothetical protein FLJ14397 | + |
| hypothetical protein LOC729580 /// hypothetical protein LOC730672 | + |
| immunoglobulin superfamily, member 8 | + |
| histone cluster 1, H1c | + |
| metallothionein 1F | + |
| espin | + |
| hypothetical protein FLJ11286 | + |
| Kruppel-like factor 7 (ubiquitous) | + |
| Homo sapiens, clone IMAGE: 5728979, mRNA | + |
| hypothetical protein LOC153546 | + |
| Leukotriene B4 receptor | + |
| GLI-Kruppel family member GLI4 | + |
| reprimo-like | + |
| Deoxyribonuclease I | + |
| hypothetical protein LOC728473 | + |
| chromosome 17 open reading frame 28 | + |
| Transcribed locus | + |
| solute carrier family 35, member D2 | + |
| gamma-glutamyltransferase-like 3 | + |
| BAI1-associated protein 2-like 1 | + |
| naked cuticle homolog 2 (Drosophila) | + |
| CDNA FLJ38785 fis, clone LIVER2001329 | + |
| histamine N-methyltransferase | + |
| RAB24, member RAS oncogene family | + |
| protease, serine, 8 | + |
| son of sevenless homolog 2 (Drosophila) | + |
| TP53 activated protein 1 | + |
| FERM, RhoGEF and pleckstrin domain protein 2 | + |
| fer-1-like 4 (C. elegans) | + |
| WD repeat domain, phosphoinositide interacting 1 | + |
| transmembrane protease, serine 3 | + |
| FXYD domain containing ion transport regulator 3 | + |
| hypothetical gene CG018 | + |
| Lck interacting transmembrane adaptor 1 | + |
| adaptor-related protein complex 1, gamma 2 subunit | + |
| insulin receptor | + |
| PP12104 | + |
| KIAA0500 protein | + |
| Transcribed locus | + |
| transcription factor 2, hepatic; LF-B3; variant hepatic nuclear factor | + |
| melanoma antigen family D, 2 | + |
| hypothetical protein KIAA1434 | + |
| MCF.2 cell line derived transforming sequence-like | + |
| Full-length cDNA clone CS0DI001YP15 of Placenta Cot 25-normalized of | + |
| Homo sapiens (human) | |
| RAB30, member RAS oncogene family | + |
| Cytochrome P450, family 3, subfamily A, polypeptide 4 | + |
| unc-51-like kinase 3 (C. elegans) | + |
| myosin VIIB | + |
| dopamine receptor D2 | + |
| cytochrome P450, family 27, subfamily A, polypeptide 1 | + |
| annexin A6 | + |
| myeloid zinc finger 1 | + |
| 4-aminobutyrate aminotransferase | + |
| FERM domain containing 4A | + |
| interferon-stimulated transcription factor 3, gamma 48 kDa | + |
| mannosidase, alpha, class 2A, member 2 | + |
| ATP-binding cassette, sub-family G (WHITE), member 1 | + |
| transmembrane, prostate androgen induced RNA | + |
| per1-like domain containing 1 | + |
| hypothetical LOC388969 | + |
| choline dehydrogenase | + |
| A kinase (PRKA) anchor protein 13 | + |
| ankyrin repeat domain 9 | + |
| CDNA clone IMAGE: 5209417 | + |
| intestine-specific homeobox | + |
| yippee-like 2 (Drosophila) | + |
| low density lipoprotein-related protein 1 (alpha-2-macroglobulin receptor) | + |
| seizure related 6 homolog (mouse)-like 2 | + |
| cytochrome P450, family 2, subfamily S, polypeptide 1 | + |
| copine II | + |
| protein phosphatase 2 (formerly 2A), regulatory subunit B″, beta | + |
| protein phosphatase 1, regulatory (inhibitor) subunit 16A | + |
| G protein-coupled receptor 153 | + |
| solute carrier family 27 (fatty acid transporter), member 1 | + |
| calmin (calponin-like, transmembrane) | + |
| trans-golgi network protein 2 | + |
| potassium voltage-gated channel, subfamily H (eag-related), member 8 | + |
| fatty acid amide hydrolase 2 | + |
| obscurin-like 1 | + |
| activin A receptor type II-like 1 | + |
| similar to GLI-Kruppel family member HKR1 | + |
| mucin 20, cell surface associated | + |
| KIAA1618 | + |
| Pre-B-cell leukemia homeobox 1 | + |
| hypothetical protein LOC644975 | + |
| FYVE, RhoGEF and PH domain containing 5 | + |
| meteorin, glial cell differentiation regulator-like /// similar to meteorin, glial | + |
| cell differentiation regulator-like | |
| solute carrier organic anion transporter family, member 3A1 | + |
| Homeobox D8 | + |
| trefoil factor 3 (intestinal) | + |
| xanthine dehydrogenase | + |
| sphingomyelin phosphodiesterase 3, neutral membrane (neutral | + |
| sphingomyelinase II) | |
| FERM domain containing 1 | + |
| annexin A13 | + |
| advillin | + |
| SNAP25-interacting protein | + |
| Transcribed locus | + |
| PHD finger protein 1 | + |
| ATP-binding cassette, sub-family C (CFTR/MRP), member 5 | + |
| golgi autoantigen, golgin subfamily a, 8B | + |
| exocyst complex component 7 | + |
| 5′-nucleotidase, ecto (CD73) | + |
| bruno-like 5, RNA binding protein (Drosophila) | + |
| chromosome 1 open reading frame 116 | + |
| Gamma tubulin ring complex protein (76p gene) | + |
| fms-related tyrosine kinase 3 ligand | + |
| sema domain, immunoglobulin domain (Ig), transmembrane domain (TM) | + |
| and short cytoplasmic domain, (semaphorin) 4G | |
| CDNA clone IMAGE: 4862812 | + |
| FYVE, RhoGEF and PH domain containing 3 | + |
| BTB (POZ) domain containing 11 | + |
| carboxyl ester lipase (bile salt-stimulated lipase) | + |
| 4-aminobutyrate aminotransferase | + |
| Polycomb group ring finger 3 | + |
| solute carrier family 1 (glutamate transporter), member 7 | + |
| carboxylesterase 3 (brain) | + |
| Transcribed locus | + |
| hypothetical protein LOC113230 | + |
| pleckstrin homology domain containing, family G (with RhoGef domain) | + |
| member 6 | |
| occludin/ELL domain containing 1 | + |
| solute carrier family 39 (metal ion transporter), member 5 | + |
| CDNA FLJ35319 fis, clone PROST2011577 | + |
| alanine-glyoxylate aminotransferase 2-like 2 | + |
| paired immunoglobin-like type 2 receptor beta | + |
| CDNA FLJ33569 fis, clone BRAMY2010317 | + |
| carnitine palmitoyltransferase 1A (liver) | + |
| hexosaminidase (glycosyl hydrolase family 20, catalytic domain) | + |
| containing | |
| transmembrane protein 176A | + |
| RAS protein activator like 1 (GAP1 like) | + |
| ATP-binding cassette, sub-family A (ABC1), member 5 | + |
| histone cluster 1, H2ae | + |
| Trinucleotide repeat containing 9 | + |
| cytochrome P450, family 3, subfamily A, polypeptide 7 | + |
| X-prolyl aminopeptidase (aminopeptidase P) 2, membrane-bound | + |
| inositol 1,4,5-triphosphate receptor, type 2 | + |
| Transcribed locus | + |
| crystallin, mu | + |
| signal peptide peptidase-like 2B | + |
| hypothetical protein FLJ10357 | + |
| phenazine biosynthesis-like protein domain containing | + |
| hypothetical protein FLJ10916 | + |
| hypothetical LOC146439 | + |
| copper chaperone for superoxide dismutase | + |
| protein C (inactivator of coagulation factors Va and VIIIa) | + |
| microtubule-associated protein 1 light chain 3 alpha | + |
| rabaptin, RAB GTPase binding effector protein 2 | + |
| hypothetical protein LOC284033 | + |
| KIAA1641 | + |
| transient receptor potential cation channel, subfamily M, member 4 | + |
| gasdermin-like | + |
| guanylate binding protein 2, interferon-inducible | + |
| fatty acid amide hydrolase | + |
| smoothelin | + |
| Fc fragment of IgG binding protein | + |
| Transcribed locus | + |
| TBC1 domain family, member 3 /// TBC1 domain family, member 3C /// | + |
| similar to USP6 N-terminal like /// similar to TBC1 domain family member | |
| 3 (Rab GTPase-activating protein PRC17) (Prostate cancer gene 17 | |
| protein) (TRE17 alpha protein) /// similar to TBC1 domain family, member 3 | |
| FLJ00299 protein | + |
| WNK lysine deficient protein kinase 2 | + |
| mucin-like protocadherin | + |
| cadherin 2, type 1, N-cadherin (neuronal) | + |
| ectonucleotide pyrophosphatase/phosphodiesterase 3 | + |
| Solute carrier family 1 (glutamate/neutral amino acid transporter), | + |
| member 4 | |
| B-cell linker | + |
| Transcribed locus | + |
| Dehydrogenase/reductase (SDR family) member 12 | + |
| SRY (sex determining region Y)-box 4 | + |
| PDZ domain containing 3 | + |
| cytochrome P450, family 3, subfamily A, polypeptide 5 | + |
| chromosome 10 open reading frame 11 | + |
| Transcribed locus | + |
| hypothetical protein LOC170425 | + |
| phosphorylase kinase, alpha 2 (liver) | + |
| transmembrane protease, serine 13 | + |
| SH3 and multiple ankyrin repeat domains 2 | + |
| protein phosphatase 1, regulatory (inhibitor) subunit 14A | + |
| nei endonuclease VIII-like 1 (E. coli) | + |
| hypothetical protein KIAA1833 /// similar to c11.1 CG12132-PA | + |
| CDNA FLJ36097 fis, clone TESTI2020956 | + |
| leukocyte-derived arginine aminopeptidase | + |
| CDNA FLJ11723 fis, clone HEMBA1005314 | + |
| enoyl Coenzyme A hydratase domain containing 2 | + |
| potassium intermediate/small conductance calcium-activated channel, | + |
| subfamily N, member 4 | |
| CDNA clone IMAGE: 5274141 | + |
| RUN and TBC1 domain containing 1 | + |
| coagulation factor X | + |
| tumor protein p53 inducible nuclear protein 2 | + |
| G protein-coupled receptor 30 | + |
| butyrophilin-like 9 | + |
| reticulocalbin 3, EF-hand calcium binding domain | + |
| transcription elongation factor A (SII), 2 | + |
| solute carrier family 44, member 1 | + |
| retinol dehydrogenase 5 (11-cis/9-cis) | + |
| calpain 13 | + |
| EGF-like-domain, multiple 8 | + |
| stimulated by retinoic acid gene 6 homolog (mouse) | + |
| coiled-coil domain containing 88B | + |
| homeobox D9 | + |
| myosin XVB pseudogene | + |
| chemokine (C-C motif) ligand 28 | + |
| leucine zipper transcription regulator 2 | + |
| Homo sapiens, Similar to hypothetical protein PRO1722, clone | + |
| IMAGE: 3342760, mRNA | |
| family with sequence similarity 3, member D | + |
| EF-hand calcium binding domain 4A | + |
| ring finger protein 186 | + |
| CDNA FLJ11723 fis, clone HEMBA1005314 | + |
| villin-like | + |
| Transcribed locus | + |
| superoxide dismutase 3, extracellular | + |
| non-metastatic cells 3, protein expressed in | + |
| cyclin-dependent kinase 3 | + |
| neurexin 2 | + |
| hypothetical protein FLJ20920 | + |
| MRNA; cDNA DKFZp686N0886 (from clone DKFZp686N0886) | + |
| chemokine (C motif) ligand 1 /// chemokine (C motif) ligand 2 | + |
| phosphatidylinositol glycan anchor biosynthesis, class Z | + |
| ubiquitin-activating enzyme E1-like | + |
| vasoactive intestinal peptide receptor 1 | + |
| Full length insert cDNA clone YQ54B06 | + |
| macrophage stimulating, pseudogene 9 | + |
| chemokine (C motif) ligand 2 | + |
| 3-hydroxy-3-methylglutaryl-Coenzyme A synthase 2 (mitochondrial) | + |
| CDNA clone IMAGE: 5274141 | + |
| KIAA0574 protein | + |
| Transcribed locus | + |
| DEAQ box polypeptide 1 (RNA-dependent ATPase) | + |
| mucin 12, cell surface associated | + |
| glycerophosphodiester phosphodiesterase domain containing 5 | + |
| ceroid-lipofuscinosis, neuronal 8 (epilepsy, progressive with mental | + |
| retardation) | |
| hypothetical protein FLJ21272 | + |
| FLJ00290 protein | + |
| solute carrier family 26 (sulfate transporter), member 2 | + |
| RASD family, member 2 | + |
| B-cell CLL/lymphoma 2 | + |
| matrilin 2 | + |
| phosphoinositide-3-kinase interacting protein 1 | + |
| flavin containing monooxygenase 5 | + |
| carcinoembryonic antigen-related cell adhesion molecule 7 | + |
| apoptosis-inducing factor, mitochondrion-associated, 3 | + |
| transmembrane protein 16J | + |
| zinc finger protein 750 | + |
| choline kinase beta /// carnitine palmitoyltransferase 1B (muscle) | + |
| Ral GEF with PH domain and SH3 binding motif 1 | + |
| KIAA1984 | + |
| kinesin family member 12 | + |
| interleukin 11 receptor, alpha | + |
| WNK lysine deficient protein kinase 4 | + |
| transformer-2 alpha | + |
| cytochrome P450, family 2, subfamily W, polypeptide 1 | + |
| spire homolog 2 (Drosophila) | + |
| lipocalcin 12 | + |
| family with sequence similarity 20, member C | + |
| transmembrane protein 63A | + |
| adenylate cyclase 4 | + |
| calcium/calmodulin-dependent protein kinase ID | + |
| deiodinase, iodothyronine, type III opposite strand | + |
| Transmembrane protein 177 | + |
| hypothetical protein FLJ33996 | + |
| cyclin J-like | + |
| yippee-like 3 (Drosophila) | + |
| Full length insert cDNA clone ZE12B03 | + |
| transforming, acidic coiled-coil containing protein 1 | + |
| chromosome 1 open reading frame 175 | + |
| sema domain, transmembrane domain (TM), and cytoplasmic domain, | + |
| (semaphorin) 6A | |
| CDNA clone IMAGE: 4346813 | + |
| kinesin family member C2 | + |
| ral guanine nucleotide dissociation stimulator-like 3 | + |
| hypothetical protein FLJ39639 | + |
| sidekick homolog 2 (chicken) | + |
| clusterin | + |
| transient receptor potential cation channel, subfamily M, member 6 | + |
| KIAA1456 protein | + |
| hydroxysteroid (17-beta) dehydrogenase 2 | + |
| phosphoenolpyruvate carboxykinase 1 (soluble) | + |
| integrin, alpha 9 | + |
| protocadherin 21 | + |
| myosin VIIA | + |
| ATPase, Class II, type 9A | + |
| Homo sapiens, clone IMAGE: 5223216 | + |
| phospholipase C, delta 1 | + |
| RAB37, member RAS oncogene family | + |
| tubulin tyrosine ligase-like family, member 3 | + |
| signal-induced proliferation-associated 1 like 2 | + |
| chromosome 6 open reading frame 123 | + |
| phospholipase A2, group X | + |
| EST from clone 208499, full insert | + |
| acyloxyacyl hydrolase (neutrophil) | + |
| transmembrane protein 44 | + |
| chromosome 10 open reading frame 10 | + |
| coagulation factor XIII, A1 polypeptide | + |
| fibronectin 1 | + |
| MRNA similar to LOC149651 (cDNA clone MGC: 39393 IMAGE: 4862156) | + |
| trehalase (brush-border membrane glycoprotein) | + |
| dynein, axonemal, heavy chain 1 | + |
| Synaptotagmin XVII | + |
| CDNA FLJ39484 fis, clone PROST2014925 /// CDNA FLJ32697 fis, clone | + |
| TESTI2000372 | |
| homeobox D11 | + |
| Transcribed locus | + |
| ADAM metallopeptidase with thrombospondin type 1 motif, 6 | + |
| synuclein, alpha interacting protein (synphilin) | + |
| uromodulin-like 1 | + |
| erythropoietin receptor | + |
| Hypothetical protein LOC283070 | + |
| receptor accessory protein 1 | + |
| hypothetical protein LOC727820 | + |
| calcyphosine | + |
| Homo sapiens, clone IMAGE: 4704591///Spectrin, beta, non-erythrocytic 5 | + |
| homeobox D10 | + |
| Transcribed locus | + |
| transmembrane protein 178 | + |
| fibroblast growth factor receptor 2 (bacteria-expressed kinase, | + |
| keratinocyte growth factor receptor, craniofacial dysostosis 1, Crouzon | |
| syndrome, Pfeiffer syndrome, Jackson-Weiss syndrome) | |
| chromosome 20 open reading frame 119 | + |
| ATPase, Ca++ transporting, ubiquitous | + |
| chromosome 5 open reading frame 4 | + |
| hypothetical protein LOC283177 | + |
| amylase, alpha 1A; salivary /// amylase, alpha 1B; salivary /// amylase, | + |
| alpha 1C (salivary) /// amylase, alpha 2A (pancreatic) /// amylase, alpha | |
| 2B (pancreatic) /// similar to Pancreatic alpha-amylase precursor (PA) | |
| (1,4-alpha-D-glucan glucanohydrolase) | |
| protocadherin 21 | + |
| calpain 3, (p94) | + |
| neurofascin homolog (chicken) | + |
| microtubule associated serine/threonine kinase family member 4 | + |
| zinc finger, matrin type 1 | + |
| platelet-derived growth factor alpha polypeptide | + |
| hypothetical protein LOC285045 | + |
| hypothetical LOC642441 /// hypothetical protein LOC730256 /// | + |
| hypothetical protein LOC730257 | |
| myeloma overexpressed gene (in a subset of t(11; 14) positive multiple | ++ |
| myelomas) | |
| Transcribed locus | ++ |
| angiopoietin 2 | ++ |
| aldehyde dehydrogenase 8 family, member A1 | ++ |
| Transcribed locus | ++ |
| gastrin-releasing peptide receptor | ++ |
| gamma-aminobutyric acid (GABA) A receptor, pi | ++ |

### Example 7

### CD133⁺ and CD117⁺ Colon Tumor Cells Are Enriched for Clonogenic Growth

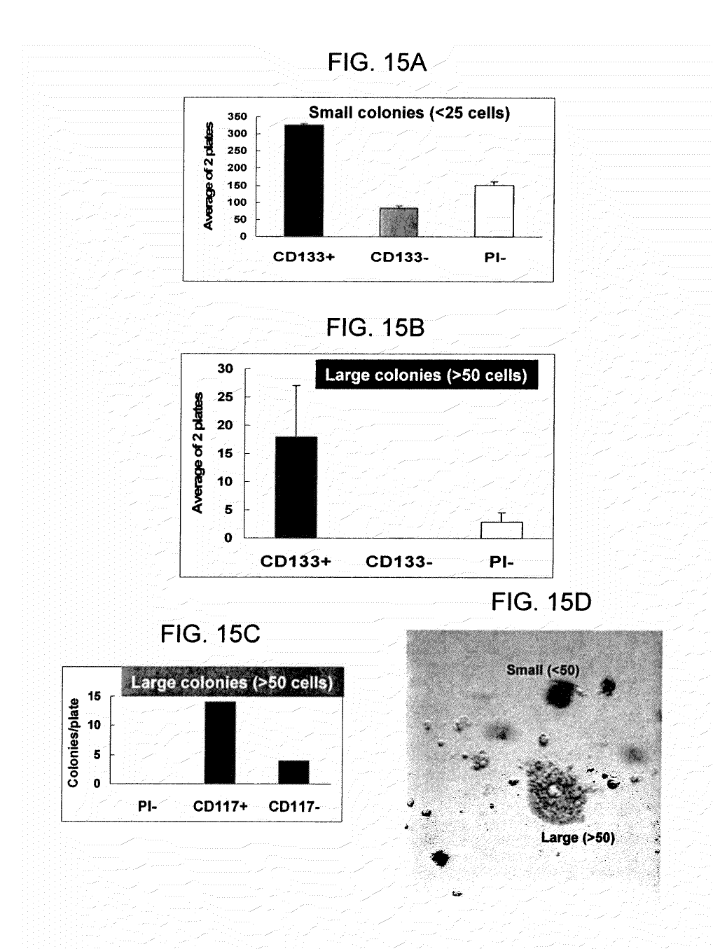

Colon tumor cells were fractionated based upon expression of CD133 or CD117 essentially as described in Example 1 and then seeded in soft agar plates. CD133⁺ cells sorted from CT1 colon tumor cells formed significantly more colonies than matched CD133⁻ cells, as shown in FIGS. 15A-15B. CD133⁺ derived colonies were also bigger than colonies derived from CD133⁻ cells (FIG. 15D), or parental unsorted cells. CD117⁺ CT1 colon tumor cells formed about 3-fold more colonies than matched CD117⁻ cells (FIG. 15C).

1. An isolated cancer stem cell population comprising at least 90% cancer stem cells, wherein the cancer stem cells (i) express ABCG2 or express CD44 at a level that is at least 5-fold greater than non-tumorigenic cells of the same origin, (ii) are tumorigenic, (iii) are capable of self-renewal, and (iv) generate tumors comprising non-tumorigenic cells.

2. The isolated cancer stem cell population of claim 1, which comprises at least 95% cancer stem cells.

3. The isolated cancer stem cell population of claim 1, wherein the cancer stem cells comprise less than about 5% of the origin tumor cell population.

4. The isolated cancer stem cell population of claim 3, wherein the cancer stem cells comprise less than about 2% of the origin tumor cell population.

5. The isolated cancer stem cell population of claim 4, wherein the cancer stem cells comprise less than about 1% of the origin tumor cell population.

6. The isolated cancer stem cell population of claim 1, wherein the cancer stem cells expressing CD44 at a level that is at least 5-fold greater than non-tumorigenic cells of the same origin comprise less than about 50% of the origin tumor cell population.

7. The isolated cancer stem cell population of claim 6, wherein the cancer stem cells expressing CD44 at a level that is at least 5-fold greater than non-tumorigenic cells of the same origin comprise less than about 33% of the origin tumor cell population.

8. The isolated cancer stem cell population of claim 7, wherein the cancer stem cells expressing CD44 at a level that is at least 5-fold greater than non-tumorigenic cells of the same origin comprise less than about 25% of the origin tumor cell population.

9. The isolated cancer stem cell population of claim 8, wherein the cancer stem cells expressing CD44 at a level that is at least 5-fold greater than non-tumorigenic cells of the same origin comprise less than about 15% of the origin tumor cell population.

10. The isolated cancer stem cell population of claim 9, wherein the cancer stem cells expressing CD44 at a level that is at least 5-fold greater than non-tumorigenic cells of the same origin comprise less than about 10% of the origin tumor cell population.

11. The isolated cancer stem cell population of claim 1, wherein the cancer stem cells additionally express β-catenin, CD117, CD133, ALDH, VLA-2, CD166, CD201, IGFR, EGF1R, or a combination thereof.

12. The isolated cancer stem cell population of claim 1, wherein the cancer stem cells do not express differentiation markers.

13. The isolated cancer stem cell population of claim 12, wherein the cancer stem cells are depleted of cells expressing CD26, Muc-1, Muc-2, villin, CD24, CEA, or CK20.

14. The isolated cancer stem cell population of claim 1, which is derived from colon.

15. The isolated cancer stem cell population of claim 1, wherein a subpopulation of about 10 cells has the capacity to form a palpable tumor.

16. An enriched cancer stem cell population derived from a tumor cell population comprising cancer stem cells and non-tumorigenic cells, wherein the cancer stem cells (i) express ABCG2 or express CD44 at a level that is at least 5-fold greater than non-tumorigenic cells of the same origin, (ii) are tumorigenic, (iii) are capable of self-renewal, (iv) generate tumors comprising non-tumorigenic cells, and (iv) are enriched at least 2-fold compared to the tumor cell population.

17. The enriched cancer stem cell population of claim 16, wherein the cancer stem cells are enriched at least 5-fold compared to tumor-derived cell population.

18. The enriched cancer stem cell population of claim 17, wherein the cancer stem cells are enriched at least 10-fold compared to tumor-derived cell population.

19. The enriched cancer stem cell population of claim 18, wherein the cancer stem cells are enriched at least 50-fold compared to tumor-derived cell population.

20. The enriched cancer stem cell population of claim 19, wherein the cancer stem cells are enriched at least 100-fold compared to tumor-derived cell population.

21. The enriched cancer stem cell population of claim 16, wherein the cancer stem cells additionally express β-catenin, CD117, CD133, ALDH, VLA-2, CD166, CD201, IGFR, EGF1R, or a combination thereof.

22. The enriched cancer stem cell population of claim 16, wherein the cancer stem cells do not express differentiation markers of the tumor cell population.

23. The enriched cancer stem cell population of claim 22, wherein the cancer stem cells are depleted of cells expressing CD26, Muc-1, Muc-2, villin, CD24, CEA, or CK20.

24. The enriched cancer stem cell population of claim 16, which is derived from colon.

25. The enriched cancer stem cell population of claim 16, wherein a subpopulation of about 10 cells has the capacity to form a palpable tumor.

26. A method of isolating a cancer stem cell population comprising:
(a) providing dissociated tumor cells, wherein a majority of the cells express CD44 at a low level, and wherein a minority of the cells express CD44 at a high level that is at least about 5-fold greater than the low level;
(b) contacting the dissociated tumor cells with an agent that specifically binds to CD44;
(c) selecting cells that specifically bind to the agent of (b) to an extent that shows a high level of CD44 expression that is at least about 5-fold greater than the low level;
whereby a cancer stem cell population is isolated.

27. The method of claim 26, wherein the cancer stem cell population comprises at least 90% cancer stem cells.

28. The method of claim 27, wherein the cancer stem cell population comprises at least 95% cancer stem cells.

29. The method of claim 26, wherein the cancer stem cell population is enriched in cancer stem cells at least 2-fold when compared to the dissociated tumor cells.

30. The method of claim 29, wherein the cancer stem cell population is enriched in cancer stem cells at least 5-fold when compared to the dissociated tumor cells.

31. The method of claim 30, wherein the cancer stem cell population is enriched in cancer stem cells at least 10-fold when compared to the dissociated tumor cells.

32. The method of claim 26, further comprising:
(d) contacting the dissociated tumor cells with an agent that specifically binds to ABCG2; and
(e) selecting cells that specifically bind to the agent of (d).

33. The method of claim 26, further comprising:
(d) contacting the dissociated tumor cells with one or more agents that specifically bind to ABCG2, CD117, CD133, ALDH, CD166, CD201, IGFR, EGF1R, or a combination thereof; and
(e) selecting cells that specifically bind to an agent or combination of agents of (d).

34. The method of claim 26, further comprising:
(d) contacting the dissociated tumor cells with one or more agents that specifically binds to a differentiation marker expressed by the tumor cells; and
(e) depleting the cancer stem cell population of cells that specifically bind to the one or more agents of (d).

35. The method of claim 34, wherein the differentiation marker is CD26.

36. The method of claim 26, wherein the agent that specifically binds CD44 is an anti-CD44 antibody.

37. The method of claim 26, wherein the selecting cells is performed by flow cytometry, fluorescence activated cell sorting, panning, affinity column separation, or magnetic selection.

38. The method of claim 26, wherein the dissociated tumor cells are colon cancer cells.

39. A cancer stem cell population isolated according to the method of claim 26.

40. A method of isolating a cancer stem cell population comprising:
(a) providing dissociated tumor cells;
(b) contacting the dissociated tumor cells with an agent that specifically binds to ABCG2;
(c) selecting cells that specifically bind to the agent of (b);
whereby a cancer stem cell population is isolated.

41. The method of claim 40, wherein the cancer stem cell population comprises at least 90% cancer stem cells.

42. The method of claim 41, wherein the cancer stem cell population comprises at least 95% cancer stem cells.

43. The method of claim 40, wherein the cancer stem cell population is enriched in cancer stem cells at least 10-fold when compared to the dissociated tumor cells.

44. The method of claim 43, wherein the cancer stem cell population is enriched in cancer stem cells at least 50-fold when compared to the dissociated tumor cells.

45. The method of claim 44, wherein the cancer stem cell population is enriched in cancer stem cells at least 100-fold when compared to the dissociated tumor cells.

46. The method of claim 40, further comprising:
(d) contacting the dissociated tumor cells with one or more agents that specifically bind to CD44, CD117, CD133, ALDH, CD166, CD201, IGFR, EGF1R, or a combination thereof; and
(e) selecting cells that specifically bind to the one or more agents of (d).

47. The method of claim 40, further comprising:
(d) contacting the dissociated tumor cells with one or more agents that specifically binds to a differentiation marker expressed by the tumor cells; and
(e) depleting the cancer stem cell population of cells that specifically bind to the one or more agents of (d).

48. The method of claim 40, wherein the differentiation marker is CD26.

49. The method of claim 40, wherein the dissociated tumor cells comprise a majority of cells expressing CD44 at a low level and a minority of cells expressing CD44 at a high level that is at least about 5-fold greater than the low level; and wherein the method further comprises:
(d) contacting the dissociated tumor cells with an agent that specifically binds to CD44; and
(e) selecting cells that bind to the agent of (d) to an extent that shows a high level of CD44 expression that is at least about 5-fold greater than the low level.

50. The method of claim 40, wherein the agent that specifically binds ABCG2 is an anti-ABCG2 antibody.

51. The method of claim 40, wherein the selecting cells is performed by flow cytometry, fluorescence activated cell sorting, panning, affinity column separation, or magnetic selection.

52. The method of claim 40, wherein the dissociated tumor cells are colon cancer cells.

53. A cancer stem cell population isolated according to the method of claim 40.

54. A method of testing efficacy of a cancer drug or candidate cancer drug comprising:
(a) providing an isolated cancer stem cell population according to claim 1;
(b) contacting the cancer stem cells with a cancer drug or a candidate cancer drug;
(c) observing a change in tumorigenic potential of the cancer stem cells following contacting the cancer stem cells with the cancer drug or candidate cancer drug.

55. A method of testing efficacy of a cancer drug or candidate cancer drug comprising:
(a) providing an enriched cancer stem cell population according to claim 16;
(b) contacting the cancer stem cells with a cancer drug or a candidate cancer drug;
(c) observing a change in tumorigenic potential of the cancer stem cells following contacting the cancer stem cells with the cancer drug or candidate cancer drug.

56. A method of testing efficacy of a cancer drug or candidate cancer drug comprising:
(a) providing a cancer stem cell population according to claim 39;
(b) contacting the cancer stem cells with a cancer drug or a candidate cancer drug;
(c) observing a change in tumorigenic potential of the cancer stem cells following contacting the cancer stem cells with the cancer drug or candidate cancer drug.

57. A method of testing efficacy of a cancer drug or candidate cancer drug comprising:
(a) providing a cancer stem cell population according to claim 53;
(b) contacting the cancer stem cells with a cancer drug or a candidate cancer drug;
(c) observing a change in tumorigenic potential of the cancer stem cells following contacting the cancer stem cells with the cancer drug or candidate cancer drug.
